(19) United States
(12) Patent Application Publication
(10) Pub. No.: US 2009/0203090 A1
(43) Pub. Date: Aug. 13, 2009

(54) METHOD FOR PRODUCING AN L-AMINO ACID USING A BACTERIUM OF THE ENTEROBACTERIACEAE FAMILY

(76) Inventors: LEONID ROMANOVICH PTITSYN, Moscow (RU); IRINA BORISOVNA ALTMAN, Moscow (RU); VERONIKA ALEKSANDROVNA KOTLIAROVA, Moscow (RU); OLGA NIKOLAEVNA MOKHOVA, Moscow (RU); TATYANA ABRAMOVNA YAMPOLSKAYA, Moscow (RU); Yury Ivanovich Kozlov, Moscow (RU); Vitaly Grigorievich Paraskevov, Moscow (RU); MASARU TERASHITA, Kawasaki-shi (JP); YOSHIHIRO USUDA, Kawasaki-shi (JP); KAZUHIKO MATSUI, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 12/354,042

(22) Filed: Jan. 15, 2009

Related U.S. Application Data
(63) Continuation of application No. PCT/JP2007/064304, filed on Jul. 12, 2007.
(60) Provisional application No. 60/885,671, filed on Jan. 19, 2007.

(30) Foreign Application Priority Data
Jul. 19, 2006 (RU) 2006125964

(51) Int. Cl. C12P 13/04; C12P 13/24; C12P 13/22; C12P 13/20; C12P 13/14; C12P 13/08; C12N 1/21

(52) U.S. Cl. 435/106; 435/107; 435/108; 435/109; 435/110; 435/115; 435/252.3; 435/252.33; 435/252.34

(57) ABSTRACT

A method for producing an L-amino acid is described, for example L-threonine, L-lysine, L-histidine, L-phenylalanine, L-arginine, L-tryptophan, or L-glutamic acid, using a bacterium of the Enterobacteriaceae family, wherein the bacterium has been modified to enhance an activity of a wild-type alcohol dehydrogenase encoded by the adhE gene or a mutant alcohol dehydrogenase which is resistant to aerobic inactivation.

This application is a continuation under 35 U.S.C. §120 to PCT Patent Application No. PCT/JP2007/064304, filed on Jul. 12, 2007, which claims priority under 35 U.S.C. §119 to Russian Patent Application No. 2006125964, filed on Jul. 19, 2006, and U.S. Provisional Patent Application No. 60/885,671, filed on Jan. 19, 2007, all of which are incorporated by reference. The Sequence Listing filed electronically herewith is also hereby incorporated by reference in its entirety (File Name: US-224_Seq_List; File Size: 109 KB; Date Created: Jan. 15, 2009).

### BACKGROUND OF THE INVENTION

1. Technical Field

The present invention relates to the microbiological industry, and specifically to a method for producing an L-amino acid such as L-threonine, L-lysine, L-histidine, L-phenylalanine, L-arginine, L-tryptophan, L-glutamic acid and L-leucine by fermentation using a bacterium with an enhanced activity of alcohol dehydrogenase.

2. Background Art

Conventionally, L-amino acids are industrially produced by fermentation methods utilizing strains of microorganisms obtained from natural sources, or mutants thereof. Typically, the microorganisms are modified to enhance production yields of L-amino acids.

Many techniques to enhance L-amino acid production yields have been reported, including transformation of microorganisms with recombinant DNA (U.S. Pat. No. 4,278,765). Other techniques for enhancing production yields include increasing the activities of enzymes involved in amino acid biosynthesis and/or desensitizing the target enzymes to feedback inhibition by the resulting L-amino acid (U.S. Pat. Nos. 4,346,170, 5,661,012, and 6,040,160).

By optimizing the main biosynthetic pathway of a desired compound, further improvement of L-amino acid producing strains can be accomplished. Typically, this is accomplished via supplementation of the bacterium with increasing amounts of a carbon source such as sugars, for example, glucose. Despite the efficiency of glucose transport by PTS, access to the carbon source in a highly productive strain still may be insufficient. Another way to increase productivity of L-amino acid producing strains and decrease the cost of the target L-amino acid is to use an alternative source of carbon, such as alcohol, for example, ethanol.

Alcohol dehydrogenase (ethanol oxidoreductase, AdhE) of Escherichia coli is a multifunctional enzyme that catalyzes fermentative production of ethanol by two sequential NADH-dependent reductions of acetyl-CoA, as well as deactivation of pyruvate formate-lyase, which cleaves pyruvate to acetyl-CoA and formate.

AdhE is abundantly synthesized (about 3×10⁴ copies per cell) during anaerobic growth in the presence of glucose and forms helical structures, called spirosomes, which are around 0.22 μm long and contain 40-60 AdhE molecules (Kessler, D., Herth, W., and Knappe, J., J. Biol. Chem., 267, 18073-18079 (1992)). When the E. coli cell culture is shifted from anaerobic to aerobic conditions, transcription of the adhE gene is reduced and maintained within 10% of the range found under anaerobiosis (Chen, Y. M., and Lin, E. C. C., J. Bacteriol. 173, 8009-8013 (1991); Leonardo, M. R., Cunningham, P. R., and Clark, D. P., J. Bacteriol. 175, 870-878 (1993); Mikulskis, A., Aristarkhov, A., and Lin, E. C. C., J. Bacteriol. 179, 7129-7134 (1997); Membrillo-Hernandez, J., and Lin, E. C. C., J. Bacteriol. 181, 7571-7579 (1999)). Translation is also regulated and requires RNase III (Membrillo-Hernandez, J., and Lin, E. C. C., J. Bacteriol. 181, 7571-7579 (1999); Aristarkhov, A. et al, J. Bacteriol. 178, 4327-4332 (1996)). AdhE has been identified as one of the major targets when E. coli cells are subjected to hydrogen peroxide stress (Tamarit, J., Cabiscol, E., and Ros, J., J. Biol. Chem. 273, 3027-3032 (1998)).

Despite the reversibility of the two NADH-coupled reactions catalyzed by AdhE, wild-type E. coli is unable to grow in the presence of ethanol as the sole source of carbon and energy, because the adhE gene is transcribed aerobically at lowered levels (Chen, Y. M. and Lin, E. C. C., J. Bacteriol. 73, 8009-8013 (1991); Leonardo, M. R., Cunningham, P. R. & Clark, D. P., J. Bacteriol. 175 870-878 (1993)) and the half-life of the AdhE protein is shortened during aerobic metabolism by metal-catalyzed oxidation (MCO).

Mutants of E. coli capable of aerobic growth on ethanol as the sole carbon and energy source have been isolated and characterized (mutants with the substitution Ala267Thr grew in the presence of ethanol with a doubling time of 240 min; with the substitutions Ala267Thr and Glu568Lys, a doubling time of 90 min at 37° C.) (Membrillo-Hernandez, J. et al, J. Biol. Chem. 275, 33869-33875 (2000); Holland-Staley, C. A. et al, J. Bacteriol. 182, 6049-6054 (2000)). Apparently, when the two sequential reactions are catalyzed in a direction opposite to that of the physiological one, acetyl-CoA formation is rate-limiting for wild-type AdhE. The tradeoff for improving the Vₘₐₓ by the A267T substitution in AdhE is decreased thermal enzyme stability and increased sensitivity to MCO damage. The second amino acid substitution, E568K, in AdhE (A267T/E568K) partially restored protein stability and resistance to MCO damage without further improvement of catalytic efficiency in substrate oxidation.

However, there have been no reports to date of using a bacterium of the Enterobacteriaceae family which has an enhanced activity of either native alcohol dehydrogenase or mutant alcohol dehydrogenase resistant to aerobic inactivation for increasing the production of L-amino acids by fermentation in a culture medium containing ethanol.

### SUMMARY OF THE INVENTION

Objects of the present invention include enhancing the productivity of L-amino acid-producing strains and providing a method for producing non-aromatic or aromatic L-amino acids using these strains.

This aim was achieved by finding that expressing either the native or mutant adhE gene which encodes alcohol dehydrogenase under the control of a promoter which functions under an aerobic cultivation condition enhances production of L-amino acids, for example, L-threonine, L-lysine, L-histidine, L-phenylalanine, L-arginine, L-tryptophan, L-glutamic acid, and/or L-leucine.

It is an aspect of the present invention to provide a method for producing an L-amino acid comprising:

A) cultivating in a culture medium containing ethanol an L-amino acid-producing bacterium of the Enterobacteriaceae family having an alcohol dehydrogenase, and

B) isolating the L-amino acid from the culture medium,

wherein the gene encoding said alcohol dehydrogenase is expressed under the control of a non-native promoter which functions under aerobic cultivation conditions.

It is a further aspect of the present invention to provide the method described above, wherein said non-native promoter is selected from the group consisting of P_{tac}, P_{lac}, P_{trP}, P_{trC}, P_{R}, and P_{L}.

It is a further aspect of the present invention to provide the method described above, wherein said alcohol dehydrogenase is resistant to aerobic inactivation.

It is a further aspect of the present invention to provide the method described above, wherein said alcohol dehydrogenase originates from a bacterium selected from the group consisting of Escherichia coli, Erwinia carotovora, Salmonella typhimurium, Shigella flexneri, Yersinia pestis, Pantoea ananatis, Lactobacillus plantarum, and Lactococcus lactis.

It is a further aspect of the present invention to provide the method described above, wherein said alcohol dehydrogenase comprises the amino acid sequence set forth in SEQ ID NO: 2, except the glutamic acid residue at position 568 is replaced with another amino acid residue other than an aspartic acid residue.

It is a further aspect of the present invention to provide the method described above, wherein said alcohol dehydrogenase comprises the amino acid sequence set forth in SEQ ID NO: 2, except the glutamic acid residue at position 568 is replaced with a lysine residue.

It is a further aspect of the present invention to provide the method described above, wherein said alcohol dehydrogenase has at least one additional mutation which is able to improve the growth of said bacterium in a liquid medium which contains ethanol as the sole carbon source.

It is a further aspect of the present invention to provide the method described above, wherein said additional mutation is selected from the group consisting of:

A) replacement of the glutamic acid residue at position 560 in SEQ ID NO: 2 with another amino acid residue;

B) replacement of the phenylalanine residue at position 566 in SEQ ID NO: 2 with another amino acid residue;

C) replacement of the glutamic acid residue, the methionine residue, the tyrosine residue, the isoleucine residue, and the alanine residue at positions 22, 236, 461, 554, and 786, respectively, in SEQ ID NO: 2 with other amino acid residues; and

D) combinations thereof.

It is a further aspect of the present invention to provide the method described above, wherein said additional mutation is selected from the group consisting of:

A) replacement of the glutamic acid residue at position 560 in SEQ ID NO: 2 with a lysine residue;

B) replacement of the phenylalanine residue at position 566 in SEQ ID NO: 2 with a valine residue;

C) replacement of the glutamic acid residue, the methionine residue, the tyrosine residue, the isoleucine residue, and the alanine residue at positions 22, 236, 461, 554, and 786 in SEQ ID NO: 2 with a glycine residue, a valine residue, a cysteine residue, a serine residue, and a valine residue, respectively; and

D) combinations thereof.

It is a further aspect of the present invention to provide the method described above, wherein said bacterium belongs to the genus selected from the group consisting of Escherichia, Enterobacter, Erwinia, Klebsiella, Pantoea, Providencia, Salmonella, Serratia, Shigella, and Morganella.

It is a further aspect of the present invention to provide the method described above, wherein said L-amino acid is selected from a group consisting of L-threonine, L-lysine, L-histidine, L-phenylalanine, L-arginine, L-tryptophan, L-glutamic acid, and L-leucine.

### BRIEF DESCRIPTION OF THE DRAWINGS

FIG. 1 shows the structure of the upstream region of the adhE gene in the chromosome of E. coli and the structure of an integrated DNA fragment containing the cat gene and a P_{L-tac}, promoter.

FIG. 2 shows the alignment of the primary sequences of alcohol dehydrogenase from Escherichia coli (ADHE_ECOLI, SEQ ID NO: 2), Shigella flexneri (Q83RN2_SHIFL, SEQ ID NO: 53), Pantoea ananatis (ADHE PANAN, SEQ ID NO: 30), Yersinia pestis (Q66AM7_YERPS, SEQ ID NO: 54), Erwinia carotovora (Q6D4R4_ERWCT, SEQ ID NO: 55), Salmonella typhimurium (P74880_SALTY, SEQ ID NO: 56), Lactobacillus plantarum (Q88RY9_LACPL, SEQ ID NO: 57) and Lactococcus lactis (O86282_{—}9LACT, SEQ ID NO: 58). The alignment was done by using the PIR Multiple Alignment program (http://pir.georgetown.edu). The identical amino acids are marked by asterisk (*), similar amino acids are marked by colon (:).

FIG. 3 shows growth curves of modified strains grown on the minimal M9 medium containing ethanol (2% or 3%) as a sole carbon source.

FIG. 4 shows growth curves of modified strains grown on the minimal M9 medium containing a mixture of glucose (0.1 weight %) and ethanol (0.1 volume %).

FIG. 5 shows comparison of growth curves of strains having mutant adhE* gene under control of the native promoter, or P_{L-tac} promoter grown on the minimal M9 medium containing ethanol (2% or 3%) as a sole carbon source.

### DETAILED DESCRIPTION OF THE PREFERRED EMBODIMENTS

Alcohol dehydrogenase is a Fe²⁺-dependent multifunctional protein with an acetaldehyde-CoA dehydrogenase activity at the N-terminal, an iron-dependent alcohol dehydrogenase activity at the C-terminal, and a pyruvate-formate lyase deactivase activity. Synonyms include B1241, AdhC, and Ana. Under aerobic conditions, the half-life of the active AdhE protein is shortened during aerobic metabolism by metal-catalyzed oxidation.

The phrase “activity of alcohol dehydrogenase” means an activity of catalyzing the reaction of NAD-dependant oxidation of alcohols into aldehydes or ketones. Alcohol dehydrogenase (EC 1.1.1.1) works well with ethanol, n-propanol, and n-butanol. Activity of alcohol dehydrogenase can be detected and measured by, for example, the method described by Membrillo-Hernandez, J. et al (J. Biol. Chem. 275, 33869-33875 (2000)).

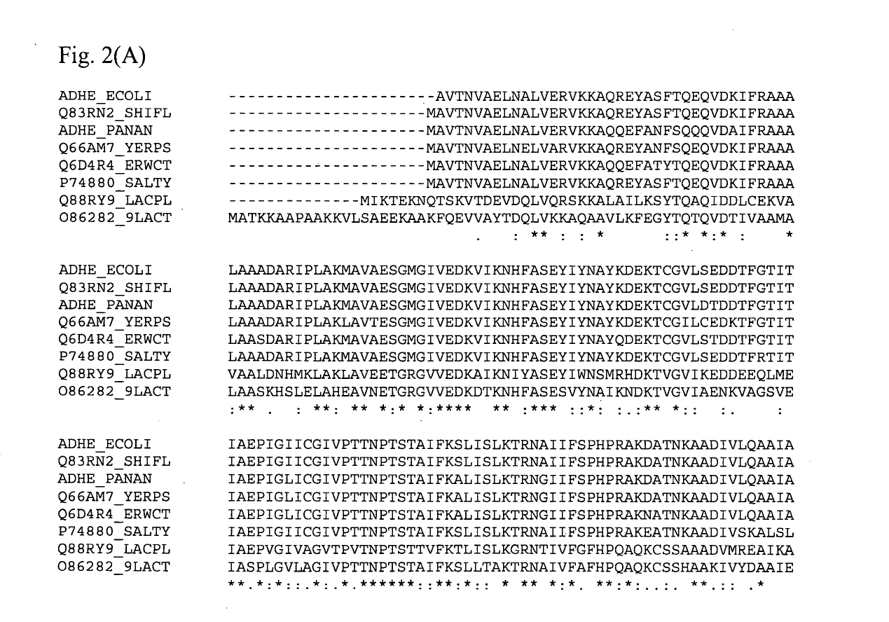

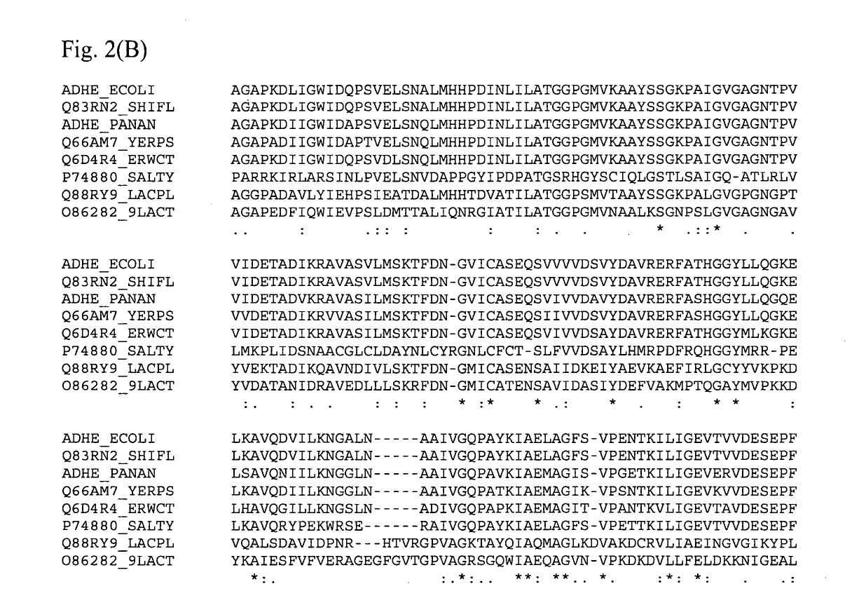

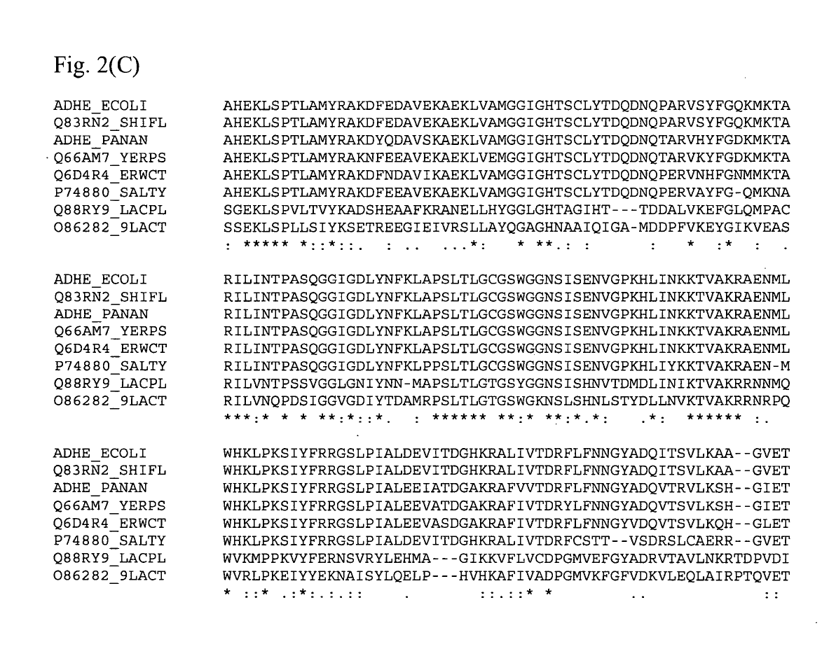

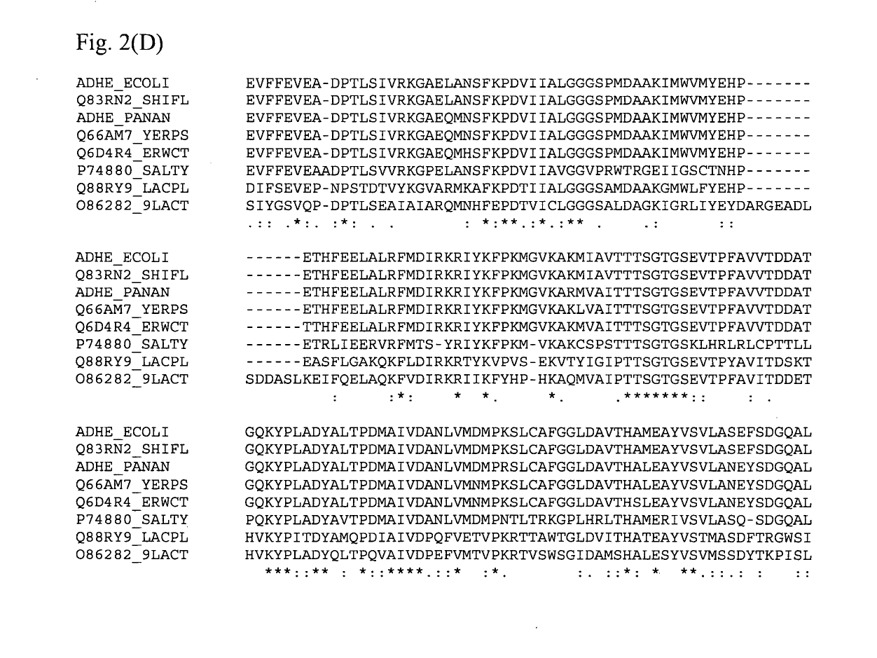

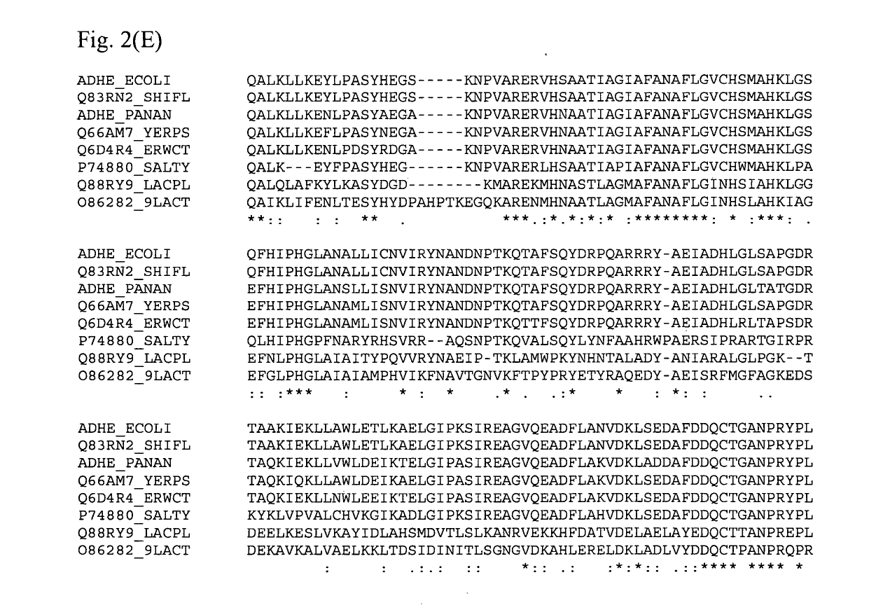

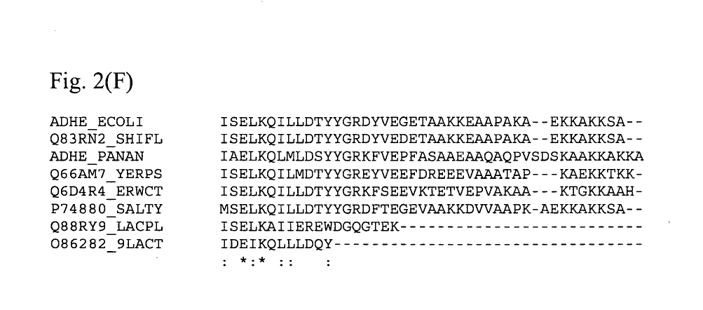

Alcohol dehydrogenase is encoded by the adhE gene, and any adhE gene derived from or native to bacteria belonging to the genus Escherichia, Erwinia, Klebsiella, Salmonella, Shigella, Yershinia, Pantoea, Lactobacillus, and Lactococcus may be used as the alcohol dehydrogenase gene. Specific examples of the source of the adhE gene include bacterial strains such as Escherichia coli, Erwinia carotovora, Salmonella enterica, Salmonella typhimurium, Shigella flexneri, Yersinia pseudotuberculosis, Pantoea ananatis, Lactobacillus plantarum and Lactococcus lactis. The wild-type adhE gene which encodes alcohol dehydrogenase from Escherichia coli has been elucidated (nucleotide numbers complementary to numbers 1294669 to 1297344 in the sequence of GenBank accession NC_{—}000913.2, gi: 49175990). The adhE gene is located between the ychG and ychE ORFs on the chromosome of E. coli K-12. Other adhE genes which encode alcohol dehydrogenases have also been elucidated: adhE gene from Erwinia carotovora (nucleotide numbers 2634501 to 2637176 in the sequence of GenBank accession NC_{—}004547.2; gi: 50121254); adhE gene from Salmonella enterica (nucleotide numbers 1718612 to 1721290 in the sequence of GenBank accession NC_{—}004631.1; gi: 29142095); adhE gene from Salmonella typhimurium (nucleotide numbers 1 to 2637 in the sequence of GenBank accession U68173.1; gi: 1519723); adhE gene from Shigella flexneri (nucleotide numbers complement to numbers 1290816 to 1293491 in the sequence of GenBank accession NC_{—}004741.1, gi: 30062760); adhE gene from Yersinia pseudotuberculosis (nucleotide numbers complement to numbers 2478099 to 2480774 in the sequence of GenBank accession NC_{—}006155.1; gi: 51596429), adhE gene from Pantoea ananatis (SEQ ID NO: 29), adhE gene from Lactobaccillus plantarum (UniProtKB Entry: Q88RY9_LACPL), adhE gene from Lactococcus lactis MG1363 (EMBL accession no. AJ001007), and the like (See FIG. 2). The nucleotide sequence of the adhE gene from Escherichia coli is represented by SEQ ID NO: 1. The amino acid sequence encoded by this adhE gene is represented by SEQ ID NO: 2.

Therefore, the adhE gene can be obtained by PCR (polymerase chain reaction; refer to White, T. J. et al., Trends Genet., 5, 185 (1989)) utilizing primers prepared based on the known nucleotide sequence of the gene from the E. coli chromosome. Genes coding for alcohol dehydrogenase from other microorganisms can be obtained in a similar manner. The adhE gene derived from Escherichia coli is exemplified by a DNA which encodes the following protein (A) or (B):

(A) a protein which has the amino acid sequence shown in SEQ ID NO: 2; or

(B) a variant protein of the amino acid sequence shown in SEQ ID NO: 2, which has an activity of alcohol dehydrogenase.

The adhE gene derived from Pantoea ananatis is exemplified by a DNA which encodes the following protein (A) or (B):

(A) a protein which has the amino acid sequence shown in SEQ ID NO: 30; or

(B) a variant protein of the amino acid sequence shown in SEQ ID NO: 30, which has an activity of alcohol dehydrogenase.

The adhE gene derived from Shigella flexneri is exemplified by a DNA which encodes the following protein (A) or (B):

(A) a protein which has the amino acid sequence shown in SEQ ID NO: 53; or

(B) a variant protein of the amino acid sequence shown in SEQ ID NO: 53, which has an activity of alcohol dehydrogenase.

The adhE gene derived from Yersinia pestis is exemplified by a DNA which encodes the following protein (A) or (B):

(A) a protein which has the amino acid sequence shown in SEQ ID NO: 54; or

(B) a variant protein of the amino acid sequence shown in SEQ ID NO: 54, which has an activity of alcohol dehydrogenase.

The adhE gene derived from Erwinia carotovora is exemplified by a DNA which encodes the following protein (A) or (B):

(A) a protein which has the amino acid sequence shown in SEQ ID NO: 55; or

(B) a variant protein of the amino acid sequence shown in SEQ ID NO: 55, which has an activity of alcohol dehydrogenase.

The adhE gene derived from Salmonella typhimurium is exemplified by a DNA which encodes the following protein (A) or (B):

(A) a protein which has the amino acid sequence shown in SEQ ID NO: 56; or

(B) a variant protein of the amino acid sequence shown in SEQ ID NO: 56, which has an activity of alcohol dehydrogenase.

The adhE gene derived from Lactobacillus plantarum is exemplified by a DNA which encodes the following protein (A) or (B):

(A) a protein which has the amino acid sequence shown in SEQ ID NO: 57; or

(B) a variant protein of the amino acid sequence shown in SEQ ID NO: 57, which has an activity of alcohol dehydrogenase.

The adhE gene derived from Lactococcus lactis is exemplified by a DNA which encodes the following protein (A) or (B):

(A) a protein which has the amino acid sequence shown in SEQ ID NO: 58; or

(B) a variant protein of the amino acid sequence shown in SEQ ID NO: 58, which has an activity of alcohol dehydrogenase.

The phrase “variant protein” means a protein which has changes in the sequence, whether they are deletions, insertions, additions, or substitutions of amino acids, but still maintains alcohol dehydrogenase activity at a useful level. The number of changes in the variant protein depends on the position in the three dimensional structure of the protein or the type of amino acid residue. The number of changes may be 1 to 30, preferably 1 to 15, and more preferably 1 to 5, relative to the protein (A). These changes in the variants are conservative mutations that preserve the function of the protein. In other words, these changes can occur in regions of the protein which are not critical for the function of the protein. This is because some amino acids have high homology to one another so the three dimensional structure or activity is not affected by such a change. Therefore, the protein variant (B) may be one which has an identity of not less than 70%, preferably not less than 80%, and more preferably not less than 90%, and most preferably not less than 95% with respect to the entire amino acid sequence of alcohol dehydrogenase shown in SEQ ID NO. 2, as long as the activity of the alcohol dehydrogenase is maintained.

Homology between two amino acid sequences can be determined using the well-known methods, for example, the computer program BLAST 2.0, which calculates three parameters: score, identity, and similarity.

The substitution, deletion, insertion, or addition of one or several amino acid residues should be conservative mutation(s) so that the activity is maintained. The representative conservative mutation is a conservative substitution. Examples of conservative substitutions include substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val.

Data comparing the primary sequences of alcohol dehydrogenase from Escherichia coli, Shigella flexneri, Pantoea ananatis, Yersinia pestis, Erwinia carotovora, Salmonella typhimurium (Gram negative bacteria), and Lactobacillus plantarum, Lactococcus lactis (Gram positive bacteria) show a high level of homology among these proteins (see FIG. 2). From this point of view, substitutions or deletions of the amino acid residues which are identical (marked by asterisk) in all the above-mentioned proteins could be crucial for their function. It is possible to replace similar (marked by colon) amino acids residues by the similar amino acid residues without deterioration of the protein activity. But modifications of other non-conserved amino acid residues may not lead to alteration of the activity of alcohol dehydrogenase.

The DNA which encodes substantially the same protein as the alcohol dehydrogenase described above may be obtained, for example, by modifying the nucleotide sequence of DNA encoding alcohol dehydrogenase (SEQ ID NO: 1), for example, by means of site-directed mutagenesis so that the nucleotide sequence responsible for one or more amino acid residues at a specified site is deleted, substituted, inserted, or added. DNA modified as described above may be obtained by conventionally known mutation treatments. Such treatments include hydroxylamine treatment of the DNA encoding proteins of present invention, or treatment of the bacterium containing the DNA with UV irradiation or a reagent such as N-methyl-N′-nitro-N-nitrosoguanidine or nitrous acid.

A DNA encoding substantially the same protein as alcohol dehydrogenase can be obtained by expressing DNA having a mutation as described above in an appropriate cell, and investigating the activity of any expressed product. A DNA encoding substantially the same protein as alcohol dehydrogenase can also be obtained by isolating a DNA that is able to hybridize with a probe having a nucleotide sequence which contains, for example, the nucleotide sequence shown as SEQ ID NO: 1, under stringent conditions, and encodes a protein having alcohol dehydrogenase activity. The “stringent conditions” referred to herein are conditions under which so-called specific hybrids are formed, and non-specific hybrids are not formed. For example, stringent conditions can be exemplified by conditions under which DNAs having high homology, for example, DNAs having identity of not less than 50%, preferably not less than 60%, more preferably not less than 70%, still more preferably not less than 80%, further preferably not less than 90%, most preferably not less than 95%, are able to hybridize with each other, but DNAs having identity lower than the above are not able to hybridize with each other, Alternatively, stringent conditions may be exemplified by conditions under which DNA is able to hybridize at a salt concentration equivalent to ordinary washing conditions in Southern hybridization, i.e., 1×SSC, 0.1% SDS, preferably 0.1×SSC, 0.1% SDS, at 60° C. Duration of washing depends on the type of membrane used for blotting and, as a rule, what is recommended by the manufacturer. For example, recommended duration of washing for the Hybond™ N+ nylon membrane (Amersham) under stringent conditions is 15 minutes. Preferably, washing may be performed 2 to 3 times.

A partial sequence of the nucleotide sequence of SEQ ID NO: 1 can also be used as a probe. Probes may be prepared by PCR using primers based on the nucleotide sequence of SEQ ID NO: 1, and a DNA fragment containing the nucleotide sequence of SEQ ID NO: 1 as a template. When a DNA fragment having a length of about 300 bp is used as the probe, the hybridization conditions for washing include, for example, 50° C., 2×SSC and 0.1% SDS.

The substitution, deletion, insertion, or addition of nucleotides as described above also includes mutations which naturally occur (mutant or variant), for example, due to variety in the species or genus of bacterium, and which contains the alcohol dehydrogenase.

A wild-type alcohol dehydrogenase may be subject to metal catalyzed oxidation. Although such a wild-type alcohol dehydrogenase can be used, a mutant alcohol dehydrogenase which is resistant to aerobic inactivation is preferable. The phrase “mutant alcohol dehydrogenase which is resistant to aerobic inactivation” means that the mutant alcohol dehydrogenase maintains its activity under aerobic conditions, or the activity is reduced by a negligible amount compared to the wild-type alcohol dehydrogenase.

In case of the adhE gene of E. coli, the wild-type alcohol dehydrogenase comprises the amino acid sequence set forth in SEQ ID NO: 2. An example of a mutation in alcohol dehydrogenase of SEQ ID NO: 2 which results in the protein being resistant to aerobic inactivation is replacement of the glutamic acid residue at position 568 with a lysine residue. However, introduction of a mutation into the adhE gene, for example at position 568 in SEQ ID NO: 2, may lead to delay of growth in a liquid medium containing ethanol as a carbon source, and in such a case, it is preferable that the mutant alcohol dehydrogenase have at least one additional mutation which is able to improve the growth of the bacterium in a liquid medium which contains ethanol as the sole carbon source. For example, the growth of E. coli is improved when the glutamic acid residue at position 568 in the alcohol dehydrogenase of SEQ ID NO: 2 is replaced by another amino acid residue by introducing an additional mutation selected from the group consisting of:

A) replacement of the glutamic acid residue at position 560 in SEQ ID NO: 2 with another amino acid residue, e.g., a lysine residue;

B) replacement of the phenylalanine residue at position 566 in SEQ ID NO: 2 with another amino acid residue, e.g., a valine residue;

C) replacement of the glutamic acid residue, the methionine residue, the tyrosine residue, the isoleucine residue, and the alanine residue at positions 22, 236, 461, 554, and 786, respectively, in SEQ ID NO: 2 with other amino acid residues, e.g., a glycine residue, a valine residue, a cysteine residue, a serine residue, and a valine residue, respectively; and

D) combinations thereof.

The reference to position numbers in a sequence, for example, the phrase “amino acid residues at positions 22, 236, 554, 560, 566, 568 and 786” refers to positions of these residues in the amino acid sequence of the wild-type AdhE from E. coli. However, the position of an amino acid residue may change. For example, if an amino acid residue is inserted at the N-terminus portion, the amino acid residue inherently located at position 22 becomes position 23. In such a case, the amino acid residue at original position 22 is the amino acid residue at position 22.

The mutant AdhE may include deletion, substitution, insertion, or addition of one or several amino acids at one or a plurality of positions other than positions identified in A) to C) above, provided that the AdhE activity is not lost or reduced.

The mutant AdhE and mutant adhE gene according to the present invention can be obtained from the wild-type adhE gene, for example, by site-specific mutagenesis using ordinary methods, such as PCR (polymerase chain reaction; refer to White, T. J. et al., Trends Genet., 5, 185 (1989)) utilizing primers prepared based on the nucleotide sequence of the gene.

Transcription of the adhE gene in wild-type E. coli is induced only under anaerobic conditions, largely in response to elevated levels of reduced NADH (Leonardo, M. R., Cunningham, P. R. & Clark, D. P., J. Bacteriol. 175 870-878 (1993)).

A bacterial strain used for producing an L-amino acid is modified so that expression of the adhE gene is controlled by a non-native promoter, i.e., a promoter that does not control the expression of the adhE gene in a wild-type strain. Such modification can be achieved by replacing the native promoter of the adhE gene on the chromosome with a non-native promoter which functions under an aerobic cultivation condition so that the adhE gene is operably linked with the non-native promoter. As a non-native promoter which functions under aerobic cultivation conditions, any promoter which can express the adhE gene above a certain level under aerobic cultivation conditions may be used. With reference to the level of the AdhE protein, the activity of alcohol dehydrogenase in the cell free extract measured according to the method by Clark and Cronan (J. Bacteriol. 141 177-183 (1980)) should be 1.5 units or more, preferably 5 units or more, and more preferably 10 units or more, per mg of protein. Aerobic cultivation conditions can be those usually used for cultivation of bacteria in which oxygen is supplied by methods such as shaking, aeration and agitation. Specifically, any promoter which is known to express a gene under aerobic cultivation conditions can be used. For example, promoters of the genes involved in glycosis, the pentose phosphate pathway, TCA cycle, amino acid biosynthetic pathways, etc. can be used. In addition, the P_{tac} promoter, the lac promoter, the trp promoter, the trc promoter, the P_{R}, or the P_{L} promoters of lambda phage are all known to be strong promoters which function under aerobic cultivation conditions, and are preferably used.

The use of a non-native promoter can be combined with the multiplication of gene copies. For example, inserting the adhE gene operably linked with a non-native promoter into a vector that is able to function in a bacterium of the Enterobacteriaceae family and introducing the vector into the bacterium increases the copy number of the gene in a cell. Preferably, low-copy vectors are used. Examples of low-copy vectors include, but are not limited to, pSC101, pMW118, pMW119, and the like. The term “low copy vector” is used for vectors, the copy number of which is up to 5 copies per cell. Increasing the copy number of the adhE gene can also be achieved by introducing multiple copies of the gene into the chromosomal DNA of the bacterium by, for example, homologous recombination, Mu integration, and the like. Homologous recombination is carried out using a sequence which is present in multiple copies as targets on the chromosomal DNA. Sequences having multiple copies on the chromosomal DNA include, but are not limited to, repetitive DNA, or inverted repeats existing at the end of a transposable element. Also, as disclosed in U.S. Pat. No. 5,595,889, it is possible to incorporate the adhE gene into a transposon, and allow it to be transferred to introduce multiple copies of the gene into the chromosomal DNA. In these instances, the adhE gene can be placed under the control of a promoter which functions under aerobic cultivation conditions. Alternatively, the effect of a promoter can be enhanced by, for example, introducing a mutation into the promoter to increase the transcription level of a gene located downstream of the promoter. Furthermore, it is known that the substitution of several nucleotides in the spacer between the ribosome binding site (RBS) and the start codon, especially the sequences immediately upstream of the start codon, profoundly affect the mRNA translatability. For example, a 20-fold range in the expression levels was found, depending on the nature of the three nucleotides preceding the start codon (Gold et al., Annu. Rev. Microbiol., 35, 365-403, 1981; Hui et al., EMBO J., 3, 623-629, 1984). Previously, it was shown that the rhtA23 mutation is an A-for-G substitution at the −1 position relative to the ATG start codon (ABSTRACTS of 17^{th} International Congress of Biochemistry and Molecular Biology in conjugation with 1997 Annual Meeting of the American Society for Biochemistry and Molecular Biology, San Francisco, Calif. Aug. 24-29, 1997, abstract No. 457). Therefore, it may be suggested that the rhtA23 mutation enhances rhtA gene expression and, as a consequence, increases resistance to threonine, homoserine, and some other substances transported out of cells.

Moreover, it is also possible to introduce a nucleotide substitution into a promoter region of the adhE gene on the bacterial chromosome, which results in stronger promoter function. The alteration of the expression control sequence can be performed, for example, in the same manner as the gene substitution using a temperature-sensitive plasmid, as disclosed in International Patent Publication WO 00/18935 and Japanese Patent Application Laid-Open No. 1-215280.

“L-amino acid-producing bacterium” means a bacterium which has an ability to produce and secrete an L-amino acid into a medium, when the bacterium is cultured in the medium. The L-amino acid-producing ability may be imparted or enhanced by breeding. The term “L-amino acid-producing bacterium” also means a bacterium which is able to produce and cause accumulation of an L-amino acid in a culture medium in an amount larger than a wild-type or parental strain of the bacterium, for example, E. coli, such as E. coli K-12, and preferably means that the bacterium is able to cause accumulation in a medium of an amount not less than 0.5 g/L, more preferably not less than 1.0 g/L of the target L-amino acid. The term “L-amino acid” includes L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine. L-threonine, L-lysine, L-histidine, L-phenylalanine, L-arginine, L-tryptophan, L-glutamic acid, and L-leucine are particularly preferred.

The Enterobacteriaceae family includes bacteria belonging to the genera Escherichia, Enterobacter, Erwinia, Klebsiella, Pantoea, Photorhabdus, Providencia, Salmonella, Serratia, Shigella, Morganella, Yersinia, etc. Specifically, those classified into the Enterobacteriaceae family according to the taxonomy used by the NCBI (National Center for Biotechnology Information) database (http://www.ncbi.nlm.nih.gov/Taxonomy/Browser/wwwtax.cgi?id=91347) can be used. A bacterium belonging to the genus Escherichia or Pantoea is preferred. The phrase “a bacterium belonging to the genus Escherichia” means that the bacterium is classified into the genus Escherichia according to the classification known to a person skilled in the art of microbiology. Examples of a bacterium belonging to the genus Escherichia include, but are not limited to, Escherichia coli (E. coli).

The bacterium belonging to the genus Escherichia that can be used is not particularly limited, however, for example, bacteria described by Neidhardt, F. C. et al. (Escherichia coli and Salmonella typhimurium, American Society for Microbiology, Washington D.C., 1208, Table 1) are encompassed by the present invention.

The bacterium belonging to the genus Pantoea means that the bacterium is classified into the genus Pantoea according to the classification known to a person skilled in the art of microbiology. Some species of Enterobacter agglomerans have been recently re-classified into Pantoea agglomerans, Pan toea ananatis, Pantoea stewartii, or the like, based on the nucleotide sequence analysis of 16S rRNA etc. (Int. J. Syst. Bacteriol., 43, 162-173 (1993)).

The bacterium of the present invention encompasses a strain of the Enterobacteriaceae family which has an ability to produce an L-amino acid and has been modified so that the gene encoding an alcohol dehydrogenase is expressed under the control of a promoter which functions under aerobic cultivation conditions. In addition, the bacterium of the present invention encompasses a strain of the Enterobacteriaceae family which has an ability to produce an L-amino acid and does not have a native activity of alcohol dehydrogenase, but has been transformed with a DNA fragment encoding alcohol dehydrogenase.

The amount of accumulated L-amino acid, for example, L-threonine, L-lysine, L-histidine, L-phenylalanine, L-arginine, L-tryptophan, L-glutamic acid, or L-leucine, can be significantly increased in a culture medium containing ethanol as a carbon source as a result of expressing the gene encoding an alcohol dehydrogenase under the control of a promoter which functions under aerobic cultivation conditions.

L-amino acid-producing bacteria

As a Bacterium of the Present Invention which is Modified to have Mutant Alcohol dehydrogenase of the present invention, bacteria which are able to produce either an aromatic or a non-aromatic L-amino acids may be used.

The bacterium of the present invention can be obtained by introducing the gene encoding the mutant alcoholdehydrogenase of the present invention in a bacterium which inherently has the ability to produce L-amino acids. Alternatively, the bacterium of present invention can be obtained by imparting the ability to produce L-amino acids to a bacterium already having the mutant alcohol dehydrogenase.

L-Threonine-Producing Bacteria

Examples of parent strains which can be used to derive the L-threonine-producing bacteria of the present invention include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli 472T23/pYN7 (ATCC 98081) (U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A), and the like.

The strain TDH-6 is deficient in the thrC gene, as well as being sucrose-assimilative, and the ilvA gene in this strain has a leaky mutation. This strain also has a mutation in the rhtA gene, which imparts resistance to high concentrations of threonine or homoserine. The strain B-3996 contains the plasmid pVIC40 which was obtained by inserting a thrA*BC operon which includes a mutant thrA gene into a RSF1010-derived vector. This mutant thrA gene encodes aspartokinase homoserine dehydrogenase I which has substantially desensitized feedback inhibition by threonine. The strain B-3996 was deposited on Nov. 19, 1987 in the All-Union Scientific Center of Antibiotics (Russia, 117105 Moscow, Nagatinskaya Street, 3-A) under the accession number RIA 1867. The strain was also deposited in the Russian National Collection of Industrial Microorganisms (VKPM) (Russia, 117545 Moscow, 1 Dorozhny proezd, 1) on Apr. 7, 1987 under the accession number VKPM B-3996.

E. coli VKPM B-5318 (EP 0593792B) also may be used as a parent strain to derive L-threonine-producing bacteria of the present invention. The strain B-5318 is prototrophic with regard to isoleucine, and a temperature-sensitive lambda-phage C1 repressor and P_{R} promoter replaces the regulatory region of the threonine operon in the plasmid pVIC40 harbored by the strain. The strain VKPM B-5318 was deposited in the Russian National Collection of Industrial Microorganisms (VKPM) on May 3, 1990 under accession number of VKPM B-5318.

Preferably, the bacterium of the present invention is additionally modified to enhance expression of one or more of the following genes:
the mutant thrA gene which codes for aspartokinase-homoserine dehydrogenase I resistant to feed back inhibition by threonine;
the thrB gene which codes for homoserine kinase;
the thrC gene which codes for threonine synthase;
the rhtA gene which codes for a putative transmembrane protein;
the asd gene which codes for aspartate-β-semialdehyde dehydrogenase; and
the aspC gene which codes for aspartate aminotransferase (aspartate transaminase);

The thrA gene which encodes aspartokinase-homoserine dehydrogenase I of Escherichia coli has been elucidated (nucleotide positions 337 to 2799, GenBank accession no.NC_{—}000913.2, gi: 49175990). The thrA gene is located between the thrL and thrB genes on the chromosome of E. coli K-12. The thrB gene which encodes homoserine kinase of Escherichia coli has been elucidated (nucleotide positions 2801 to 3733, GenBank accession NC_{—}000913.2, gi: 49175990). The thrB gene is located between the thrA and thrC genes on the chromosome of E. coli K-12. The thrC gene which encodes threonine synthase of Escherichia coli has been elucidated (nucleotide positions 3734 to 5020, GenBank accession NC_{—}000913.2, gi: 49175990). The thrC gene is located between the thrB gene and the yaaX open reading frame on the chromosome of E. coli K-12. All three genes function as a single threonine operon. To enhance expression of the threonine operon, the attenuator region which affects the transcription is desirably removed from the operon (WO2005/049808, WO2003/097839).

A mutant thrA gene which codes for aspartokinase homoserine dehydrogenase I resistant to feedback inhibition by threonine, as well as the thrB and thrC genes can be obtained as one operon from the well-known plasmid pVIC40, which is present in the threonine producing E. coli strain VKPM B-3996. Plasmid pVIC40 is described in detail in U.S. Pat. No. 5,705,371.

The rhtA gene is located at 18 min on the E. coli chromosome close to the glnHPQ operon, which encodes components of the glutamine transport system. The rhtA gene is identical to ORF1 (ybiF gene, nucleotide positions 764 to 1651, GenBank accession number AAA218541, gi:440181), and is located between the pexB and ompX genes. The DNA sequence expressing a protein encoded by the ORF1 has been designated the rhtA gene (rht: resistance to homoserine and threonine). Also, it is known that the rhtA23 mutation is an A-for-G substitution at position −1 with respect to the ATG start codon (ABSTRACTS of the 17^{th} International Congress of Biochemistry and Molecular Biology in conjugation with Annual Meeting of the American Society for Biochemistry and Molecular Biology, San Francisco, Calif. Aug. 24-29, 1997, abstract No. 457, EP 1013765 A). Hereinafter, the rhtA23 mutation is marked as rhtA*.

The asd gene of E. coli has already been elucidated (nucleotide positions 3572511 to 3571408, GenBank accession NC_{—}000913.1, gi:16131307), and can be obtained by PCR (polymerase chain reaction; refer to White, T. J. et al., Trends Genet., 5, 185 (1989)) utilizing primers prepared based on the nucleotide sequence of the gene. The asd genes of other microorganisms can be obtained in a similar manner.

Also, the aspC gene of E. coli has already been elucidated (nucleotide positions 983742 to 984932, GenBank accession NC_{—}000913.1, gi:16128895), and can be obtained by PCR. The aspC genes of other microorganisms can be obtained in a similar manner.

L-Lysine-Producing Bacteria

Examples of L-lysine-producing bacteria belonging to the genus Escherichia include mutants having resistance to an L-lysine analogue. The L-lysine analogue inhibits growth of bacteria belonging to the genus Escherichia, but this inhibition is fully or partially desensitized when L-lysine is present in the medium. Examples of the L-lysine analogue include, but are not limited to, oxalysine, lysine hydroxamate, S-(2-aminoethyl)-L-cysteine (AEC), γ-methyllysine, α-chlorocaprolactam, and so forth. Mutants having resistance to these lysine analogues can be obtained by subjecting bacteria belonging to the genus Escherichia to a conventional artificial mutagenesis treatment. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, feedback inhibition of aspartokinase by L-lysine is desensitized.

The strain WC196 may be used as an L-lysine producing bacterium of Escherichia coli. This bacterial strain was bred by conferring AEC resistance to the strain W3110, which was derived from Escherichia coli K-12. The resulting strain was designated Escherichia coli AJ13069 and was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994 and received an accession number of FERM P-14690. Then, it was converted to an international deposit under the provisions of the Budapest Treaty on Sep. 29, 1995, and received an accession number of FERM BP-5252 (U.S. Pat. No. 5,827,698).

Examples of parent strains which can be used to derive L-lysine-producing bacteria of the present invention also include strains in which expression of one or more genes encoding an L-lysine biosynthetic enzyme are enhanced. Examples of such genes include, but are not limited to, genes encoding dihydrodipicolinate synthase (dapA), aspartokinase (lysC), dihydrodipicolinate reductase (dapB), diaminopimelate decarboxylase (lysA), diaminopimelate dehydrogenase (ddh) (U.S. Pat. No. 6,040,160), phosphoenolpyrvate carboxylase (ppc), aspartate semialdehyde dehydrogenease (asd), and aspartase (aspA) (EP 1253195 A). In addition, the parent strains may have an increased level of expression of the gene involved in energy efficiency (cyo) (EP 1170376 A), the gene encoding nicotinamide nucleotide transhydrogenase (pntAB) (U.S. Pat. No. 5,830,716), the ybjE gene (WO2005/073390), or combinations thereof.

Examples of parent strains for deriving L-lysine-producing bacteria of the present invention also include strains having decreased or eliminated activity of an enzyme that catalyzes a reaction for generating a compound other than L-lysine by branching off from the biosynthetic pathway of L-lysine. Examples of the enzymes that catalyze a reaction for generating a compound other than L-lysine by branching off from the biosynthetic pathway of L-lysine include homoserine dehydrogenase, lysine decarboxylase (U.S. Pat. No. 5,827,698), and the malic enzyme (WO2005/010175).

Examples of L-lysine producing strains include E. coli WC196ΔcadAΔldc/pCABD2 (WO2006/078039). This strain was obtained by introducing the plasmid pCABD2, which is disclosed in U.S. Pat. No. 6,040,160, into the strain WC196 with the disrupted cadA and IdcC genes, which encode lysine decarboxylase. The plasmid pCABD2 contains the dapA gene of E. coli coding for a dihydrodipicolinate synthase having a mutation which desensitizes feedback inhibition by L-lysine, the lysC gene of E. coli coding for aspartokinase III having a mutation which desensitizes feedback inhibition by L-lysine, the dapB gene E. coli coding for a dihydrodipicolinate reductase, and the ddh gene of Corynebacterium glutamicum coding for diaminopimelate dehydrogenase.

L-Cysteine-Producing Bacteria

Examples of parent strains which can be used to derive L-cysteine-producing bacteria of the present invention include, but are not limited to, strains belonging to the genus Escherichia, such as E. coli JM15 which is transformed with different cysE alleles coding for feedback-resistant serine acetyltransferases (U.S. Pat. No. 6,218,168, Russian patent application 2003121601); E. coli W3110 which over-expresses genes which encode proteins suitable for secreting substances toxic for cells (U.S. Pat. No. 5,972,663); E. coli strains having lowered cysteine desulfohydrase activity (JP11155571A2); E. coli W3110 with increased activity of a positive transcriptional regulator for cysteine regulon encoded by the cysB gene (WO0127307A1), and the like.

L-Leucine-Producing Bacteria

Examples of parent strains which can be used to derive L-leucine-producing bacteria of the present invention include, but are not limited to, strains belonging to the genus Escherichia, such as E. coli strains resistant to leucine (for example, the strain 57 (VKPM B-7386, U.S. Pat. No. 6,124,121)) or leucine analogs including P3-2-thienylalanine, 3-hydroxyleucine, 4-azaleucine, 5,5,5-trifluoroleucine (JP 62-34397 B and JP 8-70879 A); E. coli strains obtained by the genetic engineering methods such as those described in WO96/06926; E. coli H-9068 (JP 8-70879 A), and the like.

The bacterium of the present invention may be improved by enhancing the expression of one or more genes involved in L-leucine biosynthesis. Examples include genes of the leuABCD operon, which are preferably represented by a mutant leuA gene coding for isopropylmalate synthase which is not subject to feedback inhibition by L-leucine (U.S. Pat. No. 6,403,342). In addition, the bacterium of the present invention may be improved by enhancing the expression of one or more genes coding for proteins which excrete L-amino acids from the bacterial cell. Examples of such genes include the b2682 and b2683 genes (ygaZH genes) (EP 1239041 A2).

L-Histidine-Producing Bacteria

Examples of parent strains which can be used to derive L-histidine-producing bacteria of the present invention include, but are not limited to, strains belonging to the genus Escherichia, such as E. coli strain 24 (VKPM B-5945, RU2003677), E. coli strain 80 (VKPM B-7270, RU2119536), E. coli NRRLB-12116-B12121 (U.S. Pat. No. 4,388,405), E. coli H-9342 (FERM BP-6675) and H-9343 (FERM BP-6676) (U.S. Pat. No. 6,344,347), E. coli H-9341 (FERM BP-6674) (EP1085087), E. coli A180/pFM201 (U.S. Pat. No. 6,258,554), and the like.

Examples of parent strains which can be used to derive L-histidine-producing bacteria of the present invention also include strains in which expression of one or more genes encoding an L-histidine biosynthetic enzyme are enhanced. Examples of such genes include genes encoding ATP phosphoribosyltransferase (hisG), phosphoribosyl AMP cyclohydrolase (hisI), phosphoribosyl-ATP pyrophosphohydrolase (hisIE), phosphoribosylformimino-5-aminoimidazole carboxamide ribotide isomerase (hisA), amidotransferase (hisH), histidinol phosphate aminotransferase (hisC), histidinol phosphatase (hisB), histidinol dehydrogenase (hisD), and so forth.

It is known that the L-histidine biosynthetic enzymes encoded by hisG and hisBHAFI are inhibited by L-histidine, and therefore an L-histidine-producing ability can also be efficiently enhanced by introducing a mutation into any of these genes which confer resistance to the feedback inhibition into enzymes encoded by the genes (Russian Patent Nos. 2003677 and 2119536).

Specific examples of strains having an L-histidine-producing ability include E. coli FERM P-5038 and 5048 which have been transformed with a vector carrying a DNA encoding an L-histidine-biosynthetic enzyme (JP 56-005099 A), E. coli strains transformed with rht, a gene for an amino acid-exporter (EP1016710A), E. coli 80 strain imparted with sulfaguanidine, DL-1,2,4-triazole-3-alanine, and streptomycin-resistance (VKPM B-7270, Russian Patent No. 2119536), and so forth.

L-Glutamic Acid-Producing Bacteria

Examples of parent strains which can be used to derive L-glutamic acid-producing bacteria of the present invention include, but are not limited to, strains belonging to the genus Escherichia, such as E. coli VL334thrC⁺ (EP 1172433). E. coli VL334 (VKPM B-1641) is an L-isoleucine and L-threonine auxotrophic strain having mutations in the thrC and ilvA genes (U.S. Pat. No. 4,278,765). A wild-type allele of the thrC gene was transferred using general transduction with a bacteriophage P1 grown on the wild-type E. coli strain K12 (VKPM B-7) cells. As a result, an L-isoleucine auxotrophic strain VL334thrC⁺ (VKPM B-8961), which is able to produce L-glutamic acid, was obtained.

Examples of parent strains which can be used to derive the L-glutamic acid-producing bacteria of the present invention include, but are not limited to, strains which are deficient in α-ketoglutarate dehydrogenase activity, or strains in which expression of one or more genes encoding an L-glutamic acid biosynthetic enzyme are enhanced. Examples of such genes include genes encoding glutamate dehydrogenase (gdh), glutamine synthetase (glnA), glutamate synthetase (gltAB), isocitrate dehydrogenase (icdA), aconitate hydratase (acnA, acnB), citrate synthase (gltA), phosphoenolpyruvate carboxylase (ppc), pyruvate dehydrogenase (aceEF, lpdA), pyruvate kinase (pykA, pykF), phosphoenolpyruvate synthase (ppsA), enolase (eno), phosphoglyceromutase (pgmA, pgmI), phosphoglycerate kinase (pgk), glyceraldehyde-3-phosphate dehydrogenase (gapA), triose phosphate isomerase (tpiA), fructose bisphosphate aldolase (fbp), phosphofructokinase (pfkA, pfkB), glucose phosphate isomerase (pgi), and so forth.

Examples of strains which have been modified so that expression of the citrate synthetase gene and/or the phosphoenolpyruvate carboxylase gene are reduced, and/or are deficient in α-ketoglutarate dehydrogenase activity include those disclosed in EP1078989A, EP955368A, and EP952221A.

Examples of parent strains which can be used to derive the L-glutamic acid-producing bacteria of the present invention also include strains having decreased or eliminated activity of an enzyme that catalyzes synthesis of a compound other than L-glutamic acid by branching off from an L-glutamic acid biosynthesis pathway. Examples of such enzymes include isocitrate lyase (aceA), α-ketoglutarate dehydrogenase (sucA), phosphotransacetylase (pta), acetate kinase (ack), acetohydroxy acid synthase (ilvG), acetolactate synthase (ilvI), formate acetyltransferase (pfl), lactate dehydrogenase (idh), and glutamate decarboxylase (gadAB). Bacteria belonging to the genus Escherichia deficient in α-ketoglutarate dehydrogenase activity or having a reduced α-ketoglutarate dehydrogenase activity and methods for obtaining them are described in U.S. Pat. Nos. 5,378,616 and 5,573,945. Specifically, these strains include the following:

E. coli W3110sucA::Km^{R}

E. coli AJ12624 (FERM BP-3853)

E. coli AJ12628 (FERM BP-3854)

E. coli AJ12949 (FERM BP-4881)

E. coli W3110sucA::Km^{R} is obtained by disrupting the α-ketoglutarate dehydrogenase gene (hereinafter referred to as “sucA gene”) of E. coli W3110. This strain is completely deficient in the α-ketoglutarate dehydrogenase activity.

Other examples of L-glutamic acid-producing bacteria include those which belong to the genus Escherichia and have resistance to an aspartic acid antimetabolite. These strains can also be deficient in the α-ketoglutarate dehydrogenase activity and include, for example, E. coli AJ13199 (FERM BP-5807) (U.S. Pat. No. 5,908,768), FFRM P-12379, which additionally has a low L-glutamic acid decomposing ability (U.S. Pat. No. 5,393,671), AJ13138 (FERM BP-5565) (U.S. Pat. No. 6,110,714), and the like.

Examples of L-glutamic acid-producing bacteria, include mutant strains belonging to the genus Pantoea which are deficient in α-ketoglutarate dehydrogenase activity or have decreased α-ketoglutarate dehydrogenase activity, and can be obtained as described above. Such strains include Pantoea ananatis AJ13356. (U.S. Pat. No. 6,331,419). Pantoea ananatis AJ13356 was deposited at the National Institute of Bioscience and Human-Technology, Agency of Industrial Science and Technology, Ministry of International Trade and Industry (currently, National Institute of Advanced Industrial Science and Technology, International Patent Organism Depositary, Central 6, 1-1, Higashi 1-Chome, Tsukuba-shi, Ibaraki-ken, 305-8566, Japan) on Feb. 19, 1998 under an accession number of FERM P-16645. It was then converted to an international deposit under the provisions of Budapest Treaty on Jan. 11, 1999 and received an accession number of FERM BP-6615. Pantoea ananatis AJ13356 is deficient in the α-ketoglutarate dehydrogenase activity as a result of disruption of the αKGDH-E1 subunit gene (sucA). The above strain was identified as Enterobacter agglomerans when it was isolated and deposited as Enterobacter agglomerans AJ13356. However, it was recently re-classified as Pantoea ananatis on the basis of nucleotide sequencing of 16S rRNA and so forth. Although AJ13356 was deposited at the aforementioned depository as Enterobacter agglomerans, for the purposes of this specification, they are described as Pantoea ananatis.

L-Phenylalanine-Producing Bacteria

Examples of parent strains which can be used to derive L-phenylalanine-producing bacteria of the present invention include, but are not limited to, strains belonging to the genus Escherichia, such as E. coli AJ12739 (tyrA::Tn10, tyrR) (VKPM B-8197), E. coli HW1089 (ATCC 55371) harboring the mutant pheA34 gene (U.S. Pat. No. 5,354,672), E. coli MWEC101-b (KR8903681), E. coli NRRB-12141, NRRB-12145, NRRB-12146 and NRRB-12147 (U.S. Pat. No. 4,407,952). Also, as a parent strain, E. coli K-12 [W3110 (tyrA)/pPHAB (FERM BP-3566), E. coli K-12 [W3110 (tyrA)/pPHAD] (FERM BP-12659), E. coli K-12 [W3110 (tyrA)/pPHATerm] (FERM BP-12662) and E. coli K-12 [W3110 (tyrA)/pBR-aroG4, pACMAB] named as AJ 12604 (FERM BP-3579) may be used (EP 488-424 B1). Furthermore, L-phenylalanine producing bacteria belonging to the genus Escherichia with an enhanced activity of the protein encoded by the yedA gene or the yddG gene may also be used (U.S. patent applications 2003/0148473 A1 and 2003/0157667 A1).

L-Tryptophan-Producing Bacteria

Examples of parent strains which can be used to derive the L-tryptophan-producing bacteria of the present invention include, but are not limited to, strains belonging to the genus Escherichia, such as E. coli JP4735/pMU3028 (DSM10122) and JP6015/pMU91 (DSM10123) which is deficient in tryptophanyl-tRNA synthetase encoded by the mutant trpS gene (U.S. Pat. No. 5,756,345), E. coli SV164 (pGH5) having a serA allele encoding phosphoglycerate dehydrogenase which is not subject to feedback inhibition by serine and a trpE allele encoding anthranilate synthase which is not subject to feedback inhibition by tryptophan (U.S. Pat. No. 6,180,373), E. coli AGX17 (pGX44) (NRRB-12263) and AGX6(pGX50) aroP (NRRB-12264) which is deficient in the enzyme tryptophanase (U.S. Pat. No. 4,371,614), E. coli AGX17/pGX50, pACKG4-pps in which a phosphoenolpyruvate-producing ability is enhanced (WO9708333, U.S. Pat. No. 6,319,696), and the like. L-tryptophan-producing bacteria belonging to the genus Escherichia which have enhanced activity of the protein encoded by the yedA or yddG genes may also be used (U.S. patent applications 2003/0148473 A1 and 2003/0157667 A1).

Examples of parent strains which can be used to derive the L-tryptophan-producing bacteria of the present invention also include strains in which one or more activities are enhanced of the following enzymes: anthranilate synthase (trpE), phosphoglycerate dehydrogenase (serA), and tryptophan synthase (trpAB). The anthranilate synthase and phosphoglycerate dehydrogenase are both subject to feedback inhibition by L-tryptophan and L-serine, therefore a mutation desensitizing the feedback inhibition may be introduced into these enzymes. Specific examples of strains having such a mutation include E. coli SV164 which harbors desensitized anthranilate synthase and a transformant strain obtained by introducing into E. coli SV164 the plasmid pGH5 (WO 94/08031), which contains a mutant serA gene encoding feedback-desensitized phosphoglycerate dehydrogenase.

Examples of parent strains which can be used to derive the L-tryptophan-producing bacteria of the present invention also include strains which have been transformed with the tryptophan operon containing a gene encoding desensitized anthranilate synthase (JP 57-71397 A, JP 62-244382 A, U.S. Pat. No. 4,371,614). Moreover, L-tryptophan-producing ability may be imparted by enhancing expression of a gene which encodes tryptophan synthase, among tryptophan operons (trpBA). Tryptophan synthase consists of a and 13 subunits which are encoded by the trpA and trpB genes, respectively. In addition, L-tryptophan-producing ability may be improved by enhancing expression of the isocitrate lyase-malate synthase operon (WO2005/103275).

L-Proline-Producing Bacteria

Examples of parent strains which can be used to derive L-proline-producing bacteria of the present invention include, but are not limited to, strains belonging to the genus Escherichia, such as E. coli 702ilvA (VKPM B-8012) which is deficient in the ilvA gene and is able to produce L-proline (EP 1172433). The bacterium of the present invention may be improved by enhancing the expression of one or more genes involved in L-proline biosynthesis. Examples of such genes include the proB gene coding for glutamate kinase which is desensitized to feedback inhibition by L-proline (DE Patent 3127361). In addition, the bacterium of the present invention may be improved by enhancing the expression of one or more genes coding for proteins responsible for secreting L-amino acids from the bacterial cell. Such genes are exemplified by the b2682 and b2683 genes (ygaZH genes) (EP1239041 A2).

Examples of bacteria belonging to the genus Escherichia, which have an activity to produce L-proline include the following E. coli strains: NRRB-12403 and NRRB-12404 (GB Patent 2075056), VKPM B-8012 (Russian patent application 2000124295), plasmid mutants described in DE Patent 3127361, plasmid mutants described by Bloom F. R. et al (The 15^{th} Miami winter symposium, 1983, p. 34), and the like.

L-Arginine-Producing Bacteria

Examples of parent strains which can be used to derive L-arginine-producing bacteria of the present invention include, but are not limited to, strains belonging to the genus Escherichia, such as E. coli strain 237 (VKPM B-7925) (U.S. Patent Application 2002/058315 A1) and derivatives thereof harboring mutant N-acetylglutamate synthase (Russian Patent Application No. 2001112869), E. coli strain 382 (VKPM B-7926) (EP1170358A1), an arginine-producing strain transformed with the argA gene encoding N-acetylglutamate synthetase (EP1170361A1), and the like.

Examples of parent strains which can be used to derive L-arginine producing bacteria of the present invention also include strains in which expression of one or more genes encoding an L-arginine biosynthetic enzyme are enhanced. Examples of such genes include genes encoding N-acetylglutamyl phosphate reductase (argC), ornithine acetyl transferase (argJ), N-acetylglutamate kinase (argB), acetylornithine transaminase (argD), ornithine carbamoyl transferase (argF), argininosuccinic acid synthetase (argG), argininosuccinic acid lyase (argH), carbamoyl phosphate synthetase (carAB), and so forth.

L-Valine-Producing Bacteria

Example of parent strains which can be used to derive L-valine-producing bacteria of the present invention include, but are not limited to, strains which have been modified to overexpress the ilvGMEDA operon (U.S. Pat. No. 5,998,178). It is desirable to remove the region of the ilvGMEDA operon responsible for attenuation so that the produced L-valine cannot attenuate expression of the operon. Furthermore, the ilvA gene in the operon is desirably disrupted so that threonine deaminase activity is decreased.

Examples of parent strains which can be used to derive L-valine-producing bacteria of the present invention also include mutants of amino-acyl t-RNA synthetase (U.S. Pat. No. 5,658,766). For example, E. coli VL1970, which has a mutation in the ileS gene encoding isoleucine tRNA synthetase, can be used. E. coli VL1970 has been deposited in the Russian National Collection of Industrial Microorganisms (VKPM) (Russia, 117545 Moscow, 1 Dorozhny Proezd, 1) on Jun. 24, 1988 under accession number VKPM B-4411.

Furthermore, mutants requiring lipoic acid for growth and/or lacking H⁺-ATPase can also be used as parent strains (WO96/06926).

L-Isoleucine-Producing Bacteria

Examples of parent strains which can be used to derive L-isoleucine producing bacteria of the present invention include, but are not limited to, mutants having resistance to 6-dimethylaminopurine (JP 5-304969 A), mutants having resistance to an isoleucine analogue such as thiaisoleucine and isoleucine hydroxamate, and mutants additionally having resistance to DL-ethionine and/or arginine hydroxamate (JP 5-130882 A). In addition, recombinant strains transformed with genes encoding proteins involved in L-isoleucine biosynthesis, such as threonine deaminase and acetohydroxate synthase, can also be used as parent strains (JP 2-458 A, FR 0356739, and U.S. Pat. No. 5,998,178).

The method for producing an L-amino acid of the present invention includes the steps of cultivating the bacterium of the present invention in a culture medium, allowing L-amino acid to accumulate in the culture medium, and collecting L-amino acid from the culture medium. Furthermore, the method of present invention includes a method for producing L-threonine, L-lysine, L-histidine, L-phenylalanine, L-arginine, L-tryptophan, L-glutamic acid, or L-leucine, including the steps of cultivating the bacterium of the present invention in a culture medium, allowing L-threonine, L-lysine, L-histidine, L-phenylalanine, L-arginine, L-tryptophan, L-glutamic acid, or L-leucine to accumulate in the culture medium, and collecting L-threonine, L-lysine, L-histidine, L-phenylalanine, L-arginine, L-tryptophan, L-glutamic acid, or L-leucine from the culture medium.

The cultivation, collection, and purification of L-amino acids from the medium and the like may be performed by conventional fermentation methods wherein an L-amino acid is produced using a bacterium.

The culture medium may be either synthetic or natural, so long as the medium includes a carbon source, a nitrogen source, minerals, and if necessary, appropriate amounts of nutrients which the bacterium requires for growth. The carbon source may include various carbohydrates such as glucose and sucrose, various organic acids and alcohols, such as ethanol. According to the present invention ethanol can be used as the sole carbon source or mixed with carbohydrates, such as glucose and sucrose. As the nitrogen source, various ammonium salts such as ammonia and ammonium sulfate, other nitrogen compounds such as amines, a natural nitrogen source such as peptone, soybean-hydrolysate, and digested fermentative microorganisms can be used. As minerals, potassium monophosphate, magnesium sulfate, sodium chloride, ferrous sulfate, manganese sulfate, calcium chloride, and the like, can be used. As vitamins, thiamine, yeast extract, and the like may be used. Additional nutrients may be added to the medium, if necessary. For example, if the bacterium requires an L-amino acid for growth (L-amino acid auxotrophy), a sufficient amount of the L-amino acid may be added to the cultivation medium.

The cultivation is preferably performed under aerobic conditions such as a shaking culture, and stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to accumulation of the target L-amino acid in the liquid medium.

After cultivation, solids such as cells can be removed from the liquid medium by centrifugation or membrane filtration, and then the target L-amino acid can be collected and purified by ion-exchange, concentration, and/or crystallization methods.

### EXAMPLES

The present invention will be more concretely explained below with reference to the following non-limiting examples.

### Example 1

### Preparation of E. Coli MG1655 Δtdh, rhtA*

The L-threonine producing E. coli strain MG1655 Atdh, rhtA* (pVIC40) was constructed by inactivation of the native tdh gene encoding threonine dehydrogenase in E. coli MG1655 (ATCC 700926) using the cat gene followed by introduction of an rhtA23 mutation (rhtA*) which confers resistance to high concentrations of threonine (>40 mg/ml) and homoserine (>5 mg/ml). Then, the resulting strain was transformed with plasmid pVIC40 from E. coli VKPM B-3996. The plasmid pVIC40 is described in detail in U.S. Pat. No. 5,705,371.

To replace the native tdh gene, a DNA fragment carrying the chloramphenicol resistance marker (Cm^{R}) encoded by the cat gene was integrated into the chromosome of E. coli MG1655 in place of the native gene by the method described by Datsenko K. A. and Wanner B. L. (Proc. Natl. Acad. Sci. USA, 2000, 97, 6640-6645) which is also called “Red-mediated integration” and/or “Red-driven integration”. The recombinant plasmid pKD46 (Datsenko, K. A., Wanner, B. L., Proc. Natl. Acad. Sci. USA, 2000, 97, 6640-6645) with the thermosensitive replicon was used as the donor of the phage λ-derived genes responsible for the Red-mediated recombination system. E. coli BW25113 containing the recombinant plasmid pKD46 can be obtained from the E. coli Genetic Stock Center, Yale University, New Haven, USA, the accession number of which is CGSC7630.

A DNA fragment containing a Cm^{R} marker encoded by the cat gene was obtained by PCR using the commercially available plasmid pACYC184 (GenBank/EMBL accession number X06403, “Fermentas”, Lithuania) as the template, and primers P1 (SEQ ID NO: 3) and P2 (SEQ ID NO: 4). Primer P1 contains 35 nucleotides homologous to the 5′-region of the tdh gene introduced into the primer for further integration into the bacterial chromosome. Primer P2 contains 32 nucleotides homologous to the 3′-region of the tdh gene introduced into the primer for further integration into the bacterial chromosome.

PCR was provided using the “Gene Amp PCR System 2700” amplificatory (Applied Biosystems). The reaction mixture (total volume—50 μl) consisted of 5 μl of 10×PCR-buffer with 25 mM MgCl₂ (“Fermentas”, Lithuania), 200 μM each of dNTP, 25 pmol each of the exploited primers and 1 U of Taq-polymerase (“Fermentas”, Lithuania). Approximately 5 ng of the plasmid DNA was added in the reaction mixture as a template DNA for the PCR amplification. The temperature profile was the following: initial DNA denaturation for 5 min at 95° C., followed by 25 cycles of denaturation at 95° C. for 30 sec, annealing at 55° C. for 30 sec, elongation at 72° C. for 40 sec; and the final elongation for 5 min at 72° C. Then, the amplified DNA fragment was purified by agarose gel-electrophoresis, extracted using “GenElute Spin Columns” (Sigma, USA), and precipitated by ethanol.

The obtained DNA fragment was used for electroporation and Red-mediated integration into the bacterial chromosome of E. coli MG1655/pKD46.

MG1655/pKD46 cells were grown overnight at 30° C. in liquid LB-medium containing ampicillin (100 μg/ml), then diluted 1:100 by SOB-medium (Yeast extract, 5 μl; NaCl, 0.5 μl; Tryptone, 20 μl; KCl, 2.5 mM; MgCl₂, 10 mM) containing ampicillin (100 μg/ml) and L-arabinose (10 mM) (arabinose is used for inducing the plasmid containing the genes of the Red system) and grown at 30° C. to reach the optical density of the bacterial culture OD₆₀₀=0.4-0.7. The grown cells from 10 ml of the bacterial culture were washed 3 times with ice-cold de-ionized water, followed by suspension in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions. Shocked cells were added to 1-ml of SOC medium (Sambrook et al, “Molecular Cloning A Laboratory Manual, Second Edition”, Cold Spring Harbor Laboratory Press (1989)), incubated for 2 hours at 37° C., and then were spread onto L-agar containing 25 μg/ml of chloramphenicol. Colonies grown for 24 hours were tested for the presence of Cm^{R} marker instead of the native tdh gene by PCR using primers P3 (SEQ ID NO: 5) and P4 (SEQ ID NO: 6). For this purpose, a freshly isolated colony was suspended in 20 μl water and then 1 μl of obtained suspension was used for PCR. The temperature profile was the following: initial DNA denaturation for 5 min at 95° C.; then 30 cycles of denaturation at 95° C. for 30 sec, annealing at 55° C. for 30 sec and elongation at 72° C. for 30 sec; the final elongation for 5 min at 72° C. A few Cm^{R} colonies tested contained the desired 1104 bp DNA fragment, confirming the presence of Cm^{R} marker DNA instead of 1242 bp fragment of tdh gene. One of the obtained strains was cured of the thermosensitive plasmid pKD46 by culturing at 37° C. and the resulting strain was named E. coli MG1655Δtdh.

Then, the rhtA23 mutation from the strain VL614rhtA23 (Livshits V. A. et al, 2003, Res. Microbiol., 154:123-135) was introduced into the obtained strain MG1655 Atdh resulting in strain MG1655 Atdh, rhtA*. The rhtA23 is a mutation which confers resistance to high concentrations of threonine (>40 mg/ml) and homoserine (>5 mg/ml). For that purpose the strain MG1655 Atdh was infected with phage Plvir grown on the donor strain VL614rhtA23. The transductants were selected on M9 minimal medium containing 8 mg/ml homoserine and 0.4% glucose as the sole carbon source.

### Example 2

### Construction of E. Coli MG1655::P_{L-tac}adhE

E. coli MG1655::P_{L-tac}adh was obtained by replacement of the native promoter region of the adhE gene in the strain MG1655 by P_{L-tac} promoter.

To replace the native promoter region of the adhE gene, the DNA fragment carrying a P_{L-tac} promoter and chloramphenicol resistance marker (Cm^{R}) encoded by the cat gene was integrated into the chromosome of E. coli MG1655 in the place of the native promoter region by the method described by Datsenko K. A. and Wanner B. L. (Proc. Natl. Acad. Sci. USA, 2000, 97, 6640-6645), which is also called “Red-mediated integration” and/or “Red-driven integration”.

A fragment containing the P_{L-tac} promoter and the cat gene was obtained by PCR using chromosomal DNA of E. coli MG1655P_{L-tac}xylE (WO2006/043730) as a template. The nucleotide sequence of the P_{L-tac} promoter is presented in the Sequence listing (SEQ ID NO: 7). Primers P5 (SEQ ID NO: 8) and P6 (SEQ ID NO: 9) were used for PCR amplification. Primer P5 contains 40 nucleotides complementary to the region located 318 bp upstream of the start codon of the adhE gene introduced into the primer for further integration into the bacterial chromosome and primer P6 contains a 39 nucleotides identical to 5′-sequence of the adhE gene.

PCR was provided using the “Gene Amp PCR System 2700” amplificatory (Applied Biosystems). The reaction mixture (total volume—50 μl) consisted of 5 μl of 10×PCR-buffer with 15 mM MgCl₂ (“Fermentas”, Lithuania), 200 μM each of dNTP, 25 pmol each of the exploited primers and 1 U of Taq-polymerase (“Fermentas”, Lithuania). Approximately 20 ng of the E. coli MG1655P_{L-tac}xylE genomic DNA was added in the reaction mixtures as a template for PCR.

The temperature profile was the following: initial DNA denaturation for 5 min at 95° C., followed by 35 cycles of denaturation at 95° C. for 30 sec, annealing at 54° C. for 30 sec, elongation at 72° C. for 1.5 min and the final elongation for 5 min at 72° C. Then, the amplified DNA fragment was purified by agarose gel-electrophoresis, extracted using “GenElute Spin Columns” (“Sigma”, USA) and precipitated by ethanol. The obtained DNA fragment was used for electroporation and Red-mediated integration into the bacterial chromosome of the E. coli MG1655/pKD46.

MG1655/pKD46 cells were grown overnight at 30° C. in the liquid LB-medium containing ampicillin (100 μg/ml), then diluted 1:100 by SOB-medium (Yeast extract, 5 μl; NaCl, 0.5 μl; Tryptone, 20 μl; KCl, 2.5 mM; MgCl₂, 10 mM) containing ampicillin (100 μg/ml) and L-arabinose (10 mM) (arabinose is used for inducing the plasmid encoding genes of the Red system) and grown at 30° C. to reach the optical density of the bacterial culture OD₆₀₀=0.4-0.7. The grown cells from 10 ml of the bacterial culture were washed 3 times with ice-cold de-ionized water, followed by suspension in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions.

Shocked cells were added to 1-ml of SOC medium (Sambrook et al, “Molecular Cloning A Laboratory Manual, Second Edition”, Cold Spring Harbor Laboratory Press (1989)), incubated for 2 hours at 37° C., and then were spread onto L-agar containing 25 μg/ml of chloramphenicol.

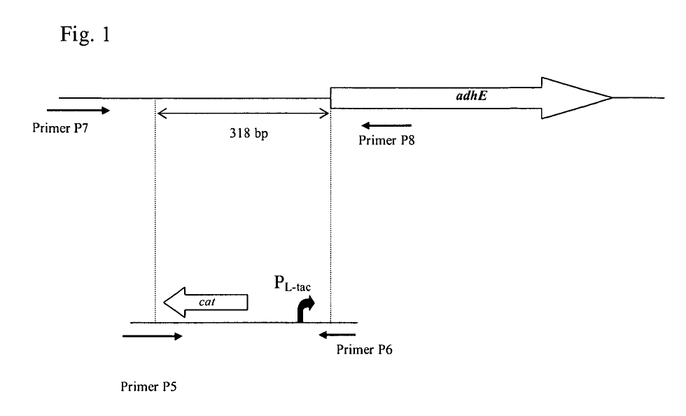

About 100 resulting clones were selected on M9 plates with 2% ethanol as the sole carbon source. Some clones which grew on M9 plates with 2% ethanol in 36 hours were chosen and tested for the presence of Cm^{R} marker instead of the native promoter region of the adhE gene by PCR using primers P7 (SEQ ID NO: 10) and P8 (SEQ ID NO: 11). For this purpose, a freshly isolated colony was suspended in 20 μl water and then 11 of the obtained suspension was used for PCR. The temperature profile follows: initial DNA denaturation for 10 min at 95° C.; then 30 cycles of denaturation at 95° C. for 30 sec, annealing at 54° C. for 30 sec and elongation at 72° C. for 1.5 min; the final elongation for 1 min at 72° C. A few Cm^{R} colonies tested contained the desired ˜1800 bp DNA fragment, confirming the presence of Cm^{R} marker DNA instead of 520 bp native promoter region of adhE gene. One of the obtained strains was cured of the thermosensitive plasmid pKD46 by culturing at 37° C. and the resulting strain was named E. coli MG 1655::P_{L-tac}adhE (See FIG. 1).

### Example 3

### Construction of E. Coli MG1655Δtdh, rhtA*, P_{L-tac}adhE

E. coli MG1655Δtdh, rhtA*, P_{L-tac}adhE was obtained by transduction of the P_{L-tac} promoter from the strain MG1655::P_{L-tac}adhE into strain MG1655Δtdh, rhtA*.

The strain MG1655Δtdh, rhtA* was infected with phage P1ᵥᵢᵣ grown on the donor strain MG1655::P_{L-tac}adhE, and the strain MG1655Δtdh, rhtA*, P_{L-tac}adhE was obtained. This strain was checked for growth on M9 plates with 2% ethanol as the sole carbon source. The growth rate was the same as for the strain MG1655::P_{L-tac}adhE.

### Example 4

### The Effect of Increasing the adhE Gene Expression on L-Threonine Production

To evaluate the effect of enhancing expression of the adhE gene on L-threonine production, both E. coli strains MG1655Δtdh, rhtA*, P_{L-tac}adhE and MG1655Δtdh, rhtA* were transformed with plasmid pVIC40.

The strain MG1655Δtdh, rhtA*, P_{L-tac}adhE (pVIC40) and a parent strain MG1655Δtdh, rhtA* (pVIC40) were each cultivated at 37° C. for 18 hours in a nutrient broth and 0.3 ml of each of the obtained cultures was inoculated into 3 ml of fermentation medium having the following composition in a 20×200 mm test tube and cultivated at 34° C. for 48 hours with a rotary shaker. Data from at least 10 independent experiments are shown on Tables 1 and 2.

Fermentation medium composition (g/l):

| Ethanol | 24 or 16 |
|---|---|
| Glucose | 0 (Table 1) or 3 (Table 2) |
| (NH₄)₂SO₄ | 16 |
| K₂HPO₄ | 0.7 |
| MgSO₄•7H₂O | 1.0 |
| MnSO₄•5H₂O | 0.01 |
| FeSO₄•7H₂O | 0.01 |
| Thiamine hydrochloride | 0.002 |
| Yeast extract | 1.0 |
| L-isoleucine | 0.01 |
| CaCO₃ | 33 |

MgSO₄.7H₂O and CaCO₃ were each sterilized separately.

It can be seen from the Tables 1 and 2, MG1655Δtdh, rhtA*, P_{L-tac}adhE was able to accumulate a higher amount of L-threonine as compared with MG1655Δtdh, rhtA*. Moreover, MG1655Δtdh, rhtA*, P_{L-tac}adhE was able to grow on the medium containing ethanol as the sole carbon source and cause accumulation of L-threonine, whereas MG1655Δtdh, rhtA* exhibited very poor growth and productivity in the medium containing ethanol as the sole carbon source.

### Example 5

### Construction of E. Coli MG1655ΔadhE

This strain was constructed by inactivation of the native adhE gene in E. coli MG1655 by the kan gene.

To inactivate (or disrupt) the native adhE gene, the DNA fragment carrying kanamycin resistance marker (Km^{R}) encoded by the kan gene was integrated into the chromosome of E. coli MG1655 (ATCC 700926) in place of the native gene by the method described by Datsenko K. A. and Wanner B. L. (Proc. Natl. Acad. Sci. USA, 2000, 97, 6640-6645) which is also called “Red-mediated integration” and/or “Red-driven integration”.

A DNA fragment containing a Km^{R} marker (kan gene) was obtained by PCR using the commercially available plasmid pACYC177 (GenBank/EMBL accession number X06402, “Fermentas”, Lithuania) as the template, and primers P9 (SEQ ID NO: 12) and P10 (SEQ ID NO: 13). Primer P9 contains 40 nucleotides homologous to the region located 318 bp upstream of the start codon of the adhE gene introduced into the primer for further integration into the bacterial chromosome. Primer P10 contains 41 nucleotides homologous to the 3′-region of the adhE gene introduced into the primer for further integration into the bacterial chromosome.

PCR was provided using the “Gene Amp PCR System 2700” amplificatory (Applied Biosystems). The reaction mixture (total volume—50 μl) consisted of 5 μl of 10×PCR-buffer with 25 mM MgCl₂ (“Fermentas”, Lithuania), 200 μM each of dNTP, 25 pmol each of the exploited primers and 1 U of Taq-polymerase (“Fermentas”, Lithuania). Approximately 5 ng of the plasmid DNA was added in the reaction mixture as a template DNA for the PCR amplification. The temperature profile was the following: initial DNA denaturation for 5 min at 95° C., followed by 25 cycles of denaturation at 95° C. for 30 sec, annealing at 55° C. for 30 sec, elongation at 72° C. for 40 sec; and the final elongation for 5 min at 72° C. Then, the amplified DNA fragment was purified by agarose gel-electrophoresis, extracted using “GenElute Spin Columns” (“Sigma”, USA) and precipitated by ethanol.

The obtained DNA fragment was used for electroporation and Red-mediated integration into the bacterial chromosome of the E. coli MG1655/pKD46.

MG1655/pKD46 cells were grown overnight at 30° C. in liquid LB-medium containing ampicillin (100 μg/ml), then diluted 1:100 by SOB-medium (Yeast extract, 5 μl; NaCl, 0.5 μl; Tryptone, 20 μl; KCl, 2.5 mM; MgCl₂, 10 mM) containing ampicillin (100 μg/ml) and L-arabinose (10 mM) (arabinose is used for inducing the plasmid encoding genes of Red system) and grown at 30° C. to reach the optical density of the bacterial culture OD₆₀₀=0.4-0.7. The grown cells from 10 ml of the bacterial culture were washed 3 times by the ice-cold de-ionized water, followed by suspension in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions. Shocked cells were added to 1-ml of SOC medium (Sambrook et al, “Molecular Cloning A Laboratory Manual, Second Edition”, Cold Spring Harbor Laboratory Press (1989)), incubated for 2 hours at 37° C., and then were spread onto L-agar containing 20 μg/ml of kanamycin. Colonies grown within 24 hours were tested for the presence of Km^{R} marker instead of the native adhE gene by PCR using primers P11 (SEQ ID NO: 14) and P12 (SEQ ID NO: 15). For this purpose, a freshly isolated colony was suspended in 20 μl water and then 1 μl of obtained suspension was used for PCR. The temperature profile follows: initial DNA denaturation for 5 min at 95° C.; then 30 cycles of denaturation at 95° C. for 30 sec, annealing at 55° C. for 30 sec and elongation at 72° C. for 30 sec; the final elongation for 5 min at 72° C. A few Km^{R} colonies tested contained the desired about 1030 bp DNA fragment, confirming the presence of Km^{R} marker DNA instead of the 3135 bp fragment of adhE gene. One of the obtained strains was cured of the thermosensitive plasmid pKD46 by culturing at 37° C. and the resulting strain was named E. coli MG1655ΔadhE.

### Example 6

### Construction of E. Coli MG1655::P_{L-tac}adhE*

E. coli MG1655::P_{L-tac}adhE* was obtained by introduction of the Glu568Lys (E568K) mutation into the adhE gene. First, 1.05 kbp fragment of the adhE gene carrying the E568K mutation was obtained by PCR using the genomic DNA of E. coli MG1655 as the template and primers P13 (SEQ ID NO: 16) and P12 (SEQ ID NO: 15). Primer P15 homologous to 1662-1701 bp and 1703-1730 bp regions of the adhE gene and includes the substitution g/a (position 1702 bp) shown as bold and primer P12 homologous to 3′-end of the adhE gene. PCR was provided using the “Gene Amp PCR System 2700” amplificatory (Applied Biosystems). The reaction mixture (total volume—50 μl) consisted of 5 μl of 10×PCR-buffer with MgCl₂ (“TaKaRa”, Japan), 250 μM each of dNTP, 25 pmol each of the exploited primers and 2.5 U of Pyrobest DNA polymerase (“TaKaRa”, Japan). Approximately 20 ng of the E. coli MG1655 genomic DNA was added in the reaction mixtures as a template for PCR. The temperature profile was the following: initial DNA denaturation for 5 min at 95° C., followed by 35 cycles of denaturation at 95° C. for 30 sec, annealing at 54° C. for 30 sec, elongation at 72° C. for 1 min and the final elongation for 5 min at 72° C. The fragment obtained was purified by agarose gel-electrophoresis, extracted using “GenElute Spin Columns” (“Sigma”, USA) and precipitated with ethanol.

In the second step, the fragment containing the P_{L-tac} promoter with the mutant adhE gene and marked by the cat gene, which provides chloramphenicol resistance, was obtained by PCR using the genomic DNA of E. coli MG1655::P_{L-tac}adhE as the template (see Example 2), primer P11 (SEQ ID NO: 14) and a 1.05 kbp fragment carrying a mutant sequence (see above) as a second primer. Primer P11 is homologous to the region located at 402-425 bp upstream of the start codon of the adhE gene. PCR was provided using the “Gene Amp PCR System 2700” amplificatory (Applied Biosystems). The reaction mixture (total volume—50 μl) consisted of 5 μl of 10×PCR-buffer (“TaKaRa”, Japan), 25 mM MgCl₂, 250 μM each of dNTP, 10 ng of the primer P11, 1 μg of the 1.05 kbp fragment as a second primer and 2.5 U of TaKaRa LA DNA polymerase (“TaKaRa”, Japan). Approximately 20 ng of the E. coli MG1655::P_{L-tac}adhE genomic DNA was added to the reaction mixture as a template for PCR. The temperature profile was the following: initial DNA denaturation for 5 min at 95° C., followed by 35 cycles of denaturation at 95° C. for 30 sec, annealing at 54° C. for 30 sec, elongation at 72° C. for 3.5 min and the final elongation for 7 min at 72° C. The resulting fragment was purified by agarose gel-electrophoresis, extracted using “GenElute Spin Columns” (“Sigma”, USA) and precipitated by ethanol.

To replace the native region of the adhE gene, the DNA fragment carrying a P_{L-tac} promoter with the mutant adhE and chloramphenicol resistance marker (Cm^{R}) encoded by the cat gene (cat-P_{L-tac}adhE*, 4.7 kbp) was integrated into the chromosome of E. coli MG1655ΔadhE by the method described by Datsenko K. A. and Wanner B. L. (Proc. Natl. Acad. Sci. USA, 2000, 97, 6640-6645) which is also called “Red-mediated integration” and/or “Red-driven integration”. MG1655 ΔadhE/pKD46 cells were grown overnight at 30° C. in liquid LB-medium containing ampicillin (100 μg/ml), then diluted 1:100 by SOB-medium (Yeast extract, 5 μl; NaCl, 0.5 μl; Tryptone, 20 μl; KCl, 2.5 mM; MgCl₂, 10 mM) containing ampicillin (100 μg/ml) and L-arabinose (10 mM) (arabinose is used for inducing the plasmid encoding genes of the Red system) and grown at 30° C. to reach the optical density of the bacterial culture OD₆₀₀=0.4-0.7. The grown cells from 10 ml of the bacterial culture were washed 3 times by the ice-cold de-ionized water, followed by suspension in 100 μl of the water. 10 μl of DNA fragment (300 ng) dissolved in the de-ionized water was added to the cell suspension. The electroporation was performed by “Bio-Rad” electroporator (USA) (No. 165-2098, version 2-89) according to the manufacturer's instructions.

Shocked cells were added to 1-ml of SOC medium (Sambrook et al, “Molecular Cloning A Laboratory Manual, Second Edition”, Cold Spring Harbor Laboratory Press (1989)), incubated for 2 hours at 37° C., and then were spread onto L-agar containing 25 μg/ml of chloramphenicol.

The clones obtained were selected on M9 plates with 2% ethanol as the sole carbon source.

The runaway clone was chosen and the full gene sequence was verified. The row of mutations was revealed as follows: Glu568Lys (gag-aag), Ile554Ser (atc- agc), Glu22Gly (gaa-gga), Met236Val (atg-gtg), Tyr461Cys (tac-tgc), Ala786Val (gca-gta). This clone was named MG1655::P_{L-tac}adhE*.

### Example 7

### Construction of E. Coli MG1655Δtdh, rhtA*, P_{L-tac}adhE*

E. coli MG1655Δtdh, rhtA*, P_{L-tac}adhE* was obtained by transduction of the P_{L-tac} adhE* mutation from the strain MG1655::P_{L-tac}adhE*.

The strain MG1655Δtdh, rhtA* was infected with phage Plvir grown on the donor strain MG1655::P_{L-tac}adhE* and the strain MG1655Δtdh, rhtA*, P_{L-tac}adhE* was obtained. This strain was checked for growth on M9 plates with 2% ethanol as a sole carbon source. The growth rate was the same as for the strain MG1655::P_{L-tac}adhE*.

### Example 8

### Construction of E. Coli MG1655Δtdh, rhtA*, P_{L-tac}adhE-Lys568

A second attempt to obtain a single mutant adhE having the Glu568Lys mutation was performed. For that purpose E. coli strain MG1655Δtdh, rhtA*, P_{L-tac}adhE-wtΔ34 was constructed.

E. coli MG1655Δtdh, rhtA*, P_{L-tac}adhE-wtΔ34 was obtained by replacement of a 34 bp fragment of the adhE gene (the region from 1668 to 1702 bp, inclusive of the triplet encoding Glu568) in E. coli MG1655Δtdh, rhtA*, P_{L-tac}adhE-wt (wt means a wild type) with kan gene. The kan gene was integrated into the chromosome of E. coli MG1655Δtdh, rhtA*, P_{L-tac}adhE-wt by the method, described by Datsenko K. A. and Wanner B. L. (Proc.Natl.Acad.Sci.USA, 2000, 97, 6640-6645) which is also called “Red-mediated integration” and/or “Red-driven integration”.

A DNA fragment containing a Km^{R} marker encoded by the kan gene was obtained by PCR using the commercially available plasmid pACYC177 (GenBank/EMBL accession number X06402, “Fermentas”, Lithuania) as the template, and primers P14 (SEQ ID NO: 17) and P15 (SEQ ID NO: 18). Primer P14 contains 41 nucleotides identical to the region from 1627 to 1668 bp of adhE gene and primer P15 contains 39 nucleotides complementary to the region from 1702 to 1740 bp of adhE gene introduced into the primers for further integration into the bacterial chromosome.

PCR was provided using the “Gene Amp PCR System 2700” amplificatory (Applied Biosystems). The reaction mixture (total volume—50 μl) consisted of 5 μl of 10×PCR-buffer with 25 mM MgCl₂ (“Fermentas”, Lithuania), 200 μM each of dNTP, 25 pmol each of the exploited primers and 1 U of Taq-polymerase (“Fermentas”, Lithuania). Approximately 5 ng of the plasmid DNA was added in the reaction mixture as a template DNA for the PCR amplification. The temperature profile was the following: initial DNA denaturation for 5 min at 95° C., followed by 25 cycles of denaturation at 95° C. for 30 sec, annealing at 55° C. for 30 sec, elongation at 72° C. 50 sec and the final elongation for 5 min at 72° C. Then, the amplified DNA fragment was purified by agarose gel-electrophoresis, extracted using “GenElute Spin Columns” (“Sigma”, USA) and precipitated by ethanol.

Colonies obtained were tested for the presence of Km^{R} marker by PCR using primers P16 (SEQ ID NO: 19) and P17 (SEQ ID NO: 20). For this purpose, a freshly isolated colony was suspended in 20 μl water and then 11 of the obtained suspension was used for PCR. The temperature profile follows: initial DNA denaturation for 5 min at 95° C.; then 30 cycles of denaturation at 95° C. for 30 sec, annealing at 55° C. for 30 sec and elongation at 72° C. for 45 sec; the final elongation for 5 min at 72° C. A few Km^{R} colonies tested contained the desired 1200 bp DNA fragment, confirming the presence of Km^{R} marker DNA instead of 230 bp fragment of native adhE gene. One of the obtained strains was cured of the thermosensitive plasmid pKD46 by culturing at 37° C. and the resulting strain was named as E. coli MG1655Δtdh, rhtA*, P_{L-tac}adhE-wtΔ34.

Then, to replace the kanamycin resistance marker (Km^{R}) encoded by kan gene with a fragment of the adhE gene encoding the Glu568Lys mutation, the oligonucleotides P18 (SEQ ID NO: 21) and P19 (SEQ ID NO: 22) carrying the appropriate mutation were integrated into the chromosome of E. coli MG1655Δtdh, rhtA, P_{L-tac}adhE-wt A34 by the method “Red-mediated integration” and/or “Red-driven integration” (Yu D., Sawitzke J. et al., Recombineering with overlapping single-stranded DNA oligonucleotides: Testing of recombination intermediate, PNAS, 2003, 100(12), 7207-7212). Primer P18 contains 75 nucleotides identical to the region from 1627 to 1702 bp of adhE gene and primer P19 contains 75 nucleotides complementary to the region from 1668 to 1740 bp of adhE gene, both primers inclusive of the triplet encoding Lys568 instead of Glu568.

The clones were selected on M9 minimal medium containing 2% ethanol and 25 mg/ml succinate as a carbon source.

Colonies were tested for the absence of Km^{R} marker by PCR using primers P16 (SEQ ID NO: 19) and P17 (SEQ ID NO: 20). For this purpose, a freshly isolated colony was suspended in 20 μl water and then 11 of the obtained suspension was used for PCR. The temperature profile follows: initial DNA denaturation for 5 min at 95° C.; then 30 cycles of denaturation at 95° C. for 30 sec, annealing at 55° C. for 30 sec and elongation at 72° C. for 25 sec; the final elongation for 5 min at 72° C. A few Km^{S} colonies tested contained the desired 230 bp DNA fragment of adhE gene, confirming the absence of Km^{R} marker DNA instead of 1200 bp fragment. Several of the obtained strains was cured of the thermosensitive plasmid pKD46 by culturing at 37° C. and the resulting strain was named as E. coli MG1655Δtdh, rhtA, P_{L-tac}adhE-Lys568.

The presence of the Glu568Lys mutation was confirmed by sequencing, for example, c1.18 has a single mutation Glu568Lys. Additionally it was found that some clones (#1, 13) contained additional mutations: cl. 1- Glu568Lys, Phe566Val; c1.13- Glu568Lys, Glu560Lys.

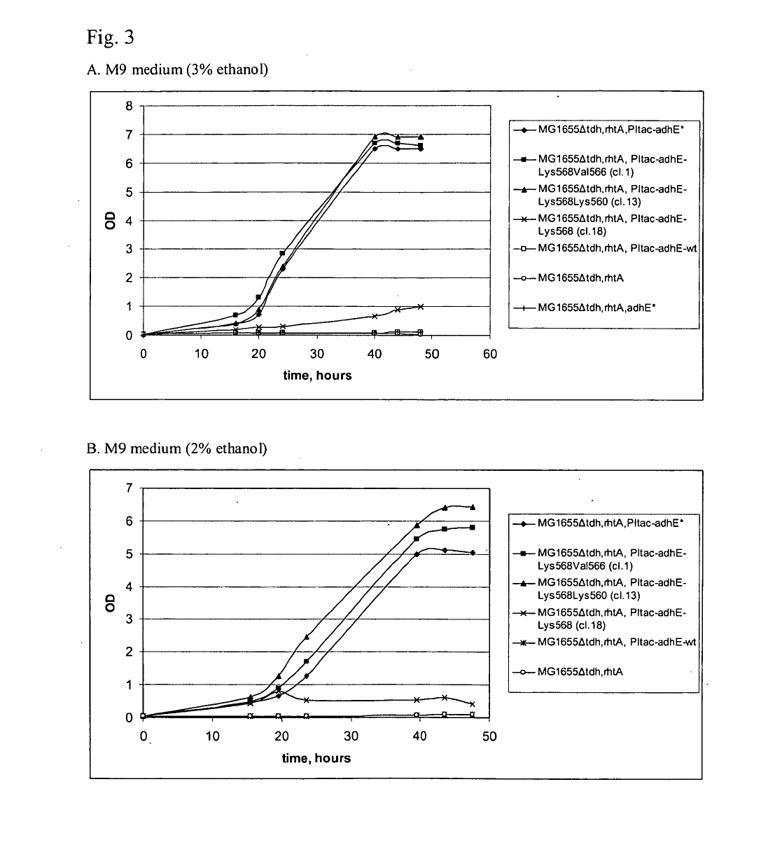

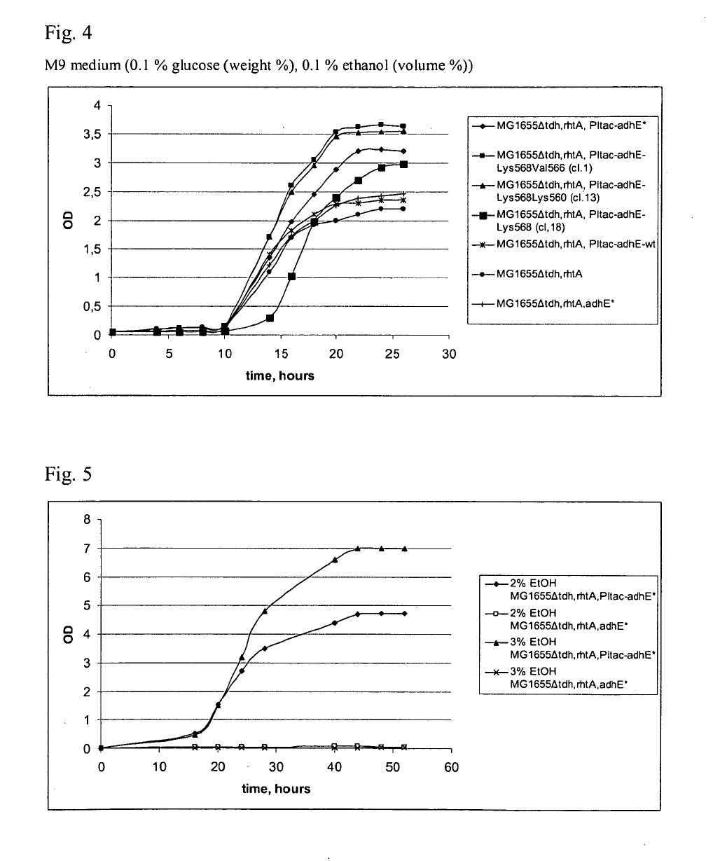

For strains MG1655Δtdh, rhtA*, P_{L-tac}adhE-Lys568 (c1.18), MG1655Δtdh, rhtA*, P_{L-tac}adhE-Lys568, Val566 (cl. 1), MG1655Δtdh, rhtA*, P_{L-tac}adhE-Lys568, Lys560 (cl. 13) and MG1655Δtdh, rhtA*, P_{L-tac}adhE*, the growth curves were studied (FIGS. 3 and 4).

The strains were grown in M9 medium with ethanol as a sole carbon source and in M9 medium with glucose and ethanol (molar ratio 1:3)

### Example 9

### Construction of E. Coli MG1655Δtdh, rhtA*, adhE*

The E. coli strain MG1655Δtdh, rhtA*, adhE* was obtained by reconstruction of the native adhE promoter in strain MG1655Δtdh, rhtA*, P_{L-tac}adhE*. A DNA fragment carrying a P_{L-tac} promoter and chloramphenicol resistance marker (Cm^{R}) encoded by cat gene in the chromosome of the strain MG1655Δtdh, rhtA*, P_{L-tac}adhE* was replaced by a fragment carrying native adhE promoter and kanamycin resistance marker (Km^{R}) encoded by the kan gene. Native P_{adhE} was obtained by PCR using a DNA of the strain MG1655 as a template and primers P20 (SEQ ID NO: 23) and P21 (SEQ ID NO: 24). Primer P20 contains an EcoRI recognition site at the 5′-end thereof, which is necessary for further joining to the kan gene and primer P21 contains 30 nucleotides homologous to 5′-region of the adhE gene (from 50 bp to 20 bp).

A DNA fragment containing a Km^{R} marker encoded by the kan gene was obtained by PCR using the commercially available plasmid pACYC177 (GenBank/EMBL accession number X06402, “Fermentas”, Lithuania) as the template, and primers P22 (SEQ ID NO: 25) and P23 (SEQ ID NO: 26). Primer P22 contains 41 nucleotides homologous to the region located 425 bp upstream of the start codon of the adhE gene introduced into the primer for further integration into the bacterial chromosome and primer P23 contains an EcoRI recognition site at the 3′-end thereof, which is necessary for further joining to the P_{adhE} promoter.

PCR were provided using the “Gene Amp PCR System 2700” amplificatory (Applied Biosystems). The reaction mixture (total volume—50 μl) consisted of 5 μl of 10×PCR-buffer with 25 mM MgCl₂ (“Fermentas”, Lithuania), 200 μM each of dNTP, 25 pmol each of the exploited primers and 1 U of Taq-polymerase (“Fermentas”, Lithuania). Approximately 20 ng of genomic DNA or 5 ng of the plasmid DNA were added in the reaction mixture as a template for the PCR amplification. The temperature profile was the following: initial DNA denaturation for 5 min at 95° C., followed by 35 cycles of denaturation for P_{adhE} or 25 cycles of denaturation for kan gene at 95° C. for 30 sec, annealing at 55° C. for 30 sec, elongation at 72° C. for 20 sec for Ptac promoter and 50 sec for kan gene; and the final elongation for 5 min at 72° C. Then, the amplified DNA fragments were purified by agarose gel-electrophoresis, extracted using “GenElute Spin Columns” (“Sigma”, USA) and precipitated by ethanol.

Each of the two above-described DNA fragments was treated with EcoRI restrictase and ligated. The ligation product was amplified by PCR using primers P21 and P22. The amplified kan-P_{adhE} DNA fragment was purified by agarose gel-electrophoresis, extracted using “GenElute Spin Columns” (“Sigma”, USA) and precipitated by ethanol. The obtained DNA fragment was used for electroporation and Red-mediated integration into the bacterial chromosome of the E. coli MG1655Δtdh::rhtA*, P_{L-tac}adhE*/pKD46.

MG1655Δtdh::rhtA*, P_{L-tac}adhE*/pKD46 cells were grown overnight at 30° C. in the liquid LB-medium with addition of ampicillin (100 μg/ml), then diluted 1:100 by the SOB-medium (Yeast extract, 5 g/l; NaCl, 0.5 g/l; Tryptone, 20 g/l; KCl, 2.5 mM; MgCl₂, 10 mM) with addition of ampicillin (100 μg/ml) and L-arabinose (10 mM) (arabinose was used for inducing the plasmid encoding genes of Red system) and grown at 30° C. to reach the optical density of the bacterial culture OD₆₀₀=0.4-0.7. The grown cells from 10 ml of the bacterial culture were washed 3 times by the ice-cold de-ionized water, followed by suspending in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions.

Shocked cells were added to 1-ml of SOC medium (Sambrook et al, “Molecular Cloning A Laboratory Manual, Second Edition”, Cold Spring Harbor Laboratory Press (1989)), incubated for 2 hours at 37° C., and then were spread onto L-agar containing 20 μg/ml of kanamycin.

Colonies grown within 24 h were tested for the presence of P_{adhE}-Km^{R} marker instead of P_{L-tac}-Cm^{R}-marker by PCR using primers P24 (SEQ ID NO: 27) and P25 (SEQ ID NO: 28). For this purpose, a freshly isolated colony was suspended in 20 μl water and then 1 μl of obtained suspension was used for PCR. The temperature profile follows: initial DNA denaturation for 5 min at 95° C.; then 30 cycles of denaturation at 95° C. for 30 sec, annealing at 54° C. for 30 sec and elongation at 72° C. for 1.0 min; the final elongation for 5 min at 72° C. A few Km^{R} colonies tested contained the desired 1200 bp DNA fragment, confirming the presence of native P_{adhE} promoter and Km^{R}-marker DNA. Some of these fragments were sequenced. The structure of the native P_{adhE} promoter was confirmed. One of the strains containing the mutant adhE gene under the control of a native promoter was cured of the thermosensitive plasmid pKD46 by culturing at 37° C. and the resulting strain was named as E. coli MG1655Δtdh, rhtA*, adhE*.

The ability of all the obtained strains MG1655Δtdh, rhtA*, P_{L-tac}adhE; MG1655Δtdh, rhtA*, P_{L-tac}adhE*; MG1655Δtdh, rhtA*, P_{L-tac}adhE-Lys568 (c1.18); MG1655Δtdh, rhtA*, P_{L-tac}adhE-Lys568, Val566 (c1.1); MG1655Δtdh, rhtA*, adhE* and parental strain MG1655Δtdh, rhtA* to grow on the minimal medium M9 containing ethanol as a sole carbon source was investigated. It was shown that the parental strain MG1655Δtdh, rhtA* and the strain with enhanced expression of wild-type alcohol dehydrogenase were unable to grow on the medium containing ethanol (2% or 3%) as a sole carbon source (FIGS. 3, A and B). Strain MG1655Δtdh, rhtA*, P_{L-tac}adhE-Lys568 (cl. 18) containing the single mutation in the alcohol dehydrogenase described early (Membrillo-Hernandez, J. et al, J. Biol. Chem. 275, 33869-33875 (2000)) exhibited very poor growth in the same medium. But strains containing mutations in the alcohol dehydrogenase in addition to mutation Glu568Lys exhibited good growth (FIGS. 3, A and B). All the above strains were able to grow on the minimal medium M9 containing a mixture of glucose and ethanol, but strains with enhanced expression of the mutant alcohol dehydrogenase containing mutations in addition to mutation Glu568Lys exhibited better growth (FIG. 4).

It was also shown that strain MG1655Δtdh, rhtA*, adhE* containing the alcohol dehydrogenase with 5 mutations under the control of the native promoter was unable to grow on the minimal medium M9 containing ethanol (2% or 3%) as a sole carbon source. Enhanced expression of the gene encoding for said alcohol dehydrogenase is necessary for good growth (FIG. 5).

### Example 10

### The Effect of Increasing the Mutant adhE Gene Expression on L-Threonine Production

To evaluate the effect of enhancing expression of the mutant adhE gene on threonine production, E. coli strains MG1655Δtdh, rhtA*, P_{L-tac}adhE; MG1655Δtdh, rhtA*, P_{L-tac}adhE*; MG1655Δtdh, rhtA*, P_{L-tac}adhE-Lys568 (cl. 18); MG1655Δtdh, rhtA*, P_{L-tac}adhE-Lys568, Val566 (cl. 1); MG1655Δtdh, rhtA*, adhE* and parental strain MG1655Δtdh, rhtA* were transformed with plasmid pVIC40.

These strains and the parent strain MG1655Δtdh, rhtA* (pVIC40) were cultivated at 37° C. for 18 hours in a nutrient broth and 0.3 ml of each of the obtained cultures was inoculated into 3 ml of fermentation medium (see Example 4) in a 20×200 mm test tube and cultivated at 34° C. for 48 hours with a rotary shaker. Data from at least 10 independent experiments are shown on Tables 1 and 2.

It can be seen from the Tables 1 and 2, mutant alcohol dehydrogenase was able to cause accumulation of a higher amount of L-threonine as compared with MG1655Δtdh, rhtA* in which neither expression of a wild-type nor a mutant alcohol dehydrogenase was increased or even with MG1655Δtdh, rhtA*, or P_{L-tac}adhE, in which expression of wild-type alcohol dehydrogenase was increased. Such higher accumulation of L-threonine during fermentation was observed in the medium containing either a mixture of glucose and ethanol, or just ethanol as the sole carbon source.

TABLE 1

| | 3% ethanol | | 2% ethanol | |
|---|---|---|---|---|
| Strain | OD₅₄₀ | Thr, g/l | OD₅₄₀ | Thr, g/l |
| MG1655Δtdh, rhtA* (pVIC40) | 1.6 ± 0.1 | <0.1 | 1.4 ± 0.1 | <0.1 |
| MG1655Δtdh, rhtA*, P_{L-tac}adhE (wt) (pVIC40) | 7.9 ± 0.3 | 1.1 ± 0.1 | 7.6 ± 0.2 | 0.9 ± 0.1 |
| MG1655Δtdh, rhtA*, | 14.7 ± 0.3 | 3.3 ± 0.1 | 13.7 ± 0.4 | 2.3 ± 0.3 |
| P_{L-tac}adhE-Lys568 (pVIC40) (cl.18) | | | | |
| MG1655Δtdh, rhtA*, P_{L-tac}adhE-Lys568, | 14.2 ± 0.4 | 3.2 ± 0.2 | 12.5 ± 0.3 | 2.1 ± 0.3 |
| Val566 (pVIC40) (cl.1) | | | | |
| MG1655Δtdh, rhtA*, P_{L-tac}adhE* (pVIC40) | 17.0 ± 0.3 | 3.9 ± 0.2 | 14.3 ± 0.3 | 2.8 ± 0.1 |
| MG1655Δtdh, rhtA*, adhE* (pVIC40) | 2.8 ± 0.2 | <0.1 | 2.1 ± 0.1 | <0.1 |

TABLE 2

| | 2.7% ethanol + 0.3% | |
|---|---|---|
| | glucose | |
| Strain | OD₅₄₀ | Thr, g/l |
| MG1655Δtdh, rhtA* (pVIC40) | 6.6 ± 0.2 | 0.9 ± 0.2 |
| MG1655Δtdh, rhtA*, P_{L-tac}adhE (wt) (pVIC40) | 13.4 ± 0.3 | 1.4 ± 0.3 |
| MG1655Δtdh, rhtA*, P_{L-tac}adhE-Lys568 | 16.1 ± 0.4 | 2.6 ± 0.2 |
| (pVIC40) (cl.18) | | |
| MG1655Δtdh, rhtA*, P_{L-tac}adhE-Lys568, | 15.5 ± 0.3 | 2.9 ± 0.2 |
| Val566(pVIC40) (cl.1) | | |
| MG1655Δtdh, rhtA*, P_{L-tac}adhE* (pVIC40) | 18.8 ± 0.4 | 2.8 ± 0.1 |
| MG1655Δtdh, rhtA*, adhE* (pVIC40) | 5.8 ± 0.1 | 0.8 ± 0.3 |

Test-tube fermentation was carried out without reversion of evaporated ethanol.

### Example 11

### The Effect of Increasing adhE Gene Expression on L-Lysine Production

To test the effect of enhanced expression of the adhE gene under the control of P_{L-tac} promoter on lysine production, the DNA fragments from the chromosome of the above-described strains MG1655Δtdh, rhtA*, P_{L-tac}adhE; MG1655Δtdh, rhtA*, P_{L-tac}adhE*; MG1655Δtdh, rhtA*, P_{L-tac}adhE-Lys568 (cl. 18); MG1655Δtdh, rhtA*, P_{L-tac}adhE-Lys568, Val566 (cl. 1); MG1655Δtdh, rhtA*, adhE* were transferred to the lysine-producing E. coli strain WC196ΔcadAΔldc (pCABD2) by P1 transduction (Miller, J. H. (1972) Experiments in Molecular Genetics, Cold Spring Harbor Lab. Press, Plainview, N.Y.). pCABD2 is a plasmid comprising the dapA gene coding for a dihydrodipicolinate synthase having a mutation which desensitizes feedback inhibition by L-lysine, the lysC gene coding for aspartokinase III having a mutation which desensitizes feedback inhibition by L-lysine, the dapB gene coding for a dihydrodipicolinate reductase, and the ddh gene coding for diaminopimelate dehydrogenase (U.S. Pat. No. 6,040,160).

The resulting strains and the parent strain WC196ΔcadAΔldc (pCABD2) were spread on L-medium plates containing 20 mg/l of streptomycin at 37° C., and cells corresponding to ⅛ of a plate were inoculated into 20 ml of the fermentation medium containing the required drugs in a 500 ml-flask. The cultivation can be carried out at 37° C. for 48 hours by using a reciprocal shaker at the agitation speed of 115 rpm. After the cultivation, the amounts of L-lysine and residual ethanol in the medium can be measured by a known method (Bio-Sensor BF-5, manufactured by Oji Scientific Instruments). Then, the yield of L-lysine relative to consumed ethanol can be calculated for each of the strains.

The composition of the fermentation medium (g/l) was as follows:

| | |
|---|---|
| Ethanol | 20.0 |
| (NH₄)₂SO₄ | 24.0 |
| K₂HPO₄ | 1.0 |
| MgSO₄•7H₂O | 1.0 |
| FeSO₄•7H₂O | 0.01 |
| MnSO₄•5H₂O | 0.01 |
| Yeast extract | 2.0 |

pH is adjusted to 7.0 by KOH and the medium was autoclaved at 115° C. for 10 min. Ethanol and MgSO₄ 7H₂O were sterilized separately. CaCO₃ was dry-heat sterilized at 180° C. for 2 hours and added to the medium at a final concentration of 30 μl. Data from two parallel experiments are shown on Table 3.

TABLE 3

| | 2% ethanol | |
|---|---|---|
| Strain | OD₆₀₀ | Lys, g/l |
| WC196ΔcadAΔldc (pCABD2) | 1.1 ± 0.0 | 0.2 ± 0.0 |
| WC196ΔcadAΔldc, P_{L-tac}adhE (wt) (pCABD2) | 1.5 ± 0.1 | 0.4 ± 0.1 |
| MG1655ΔcadAΔldc, P_{L-tac}adhE-Lys568 | 5.2 ± 0.4 | 1.3 ± 0.1 |
| (pCABD2) (cl.18) | | |
| MG1655ΔcadAΔldc, P_{L-tac}adhE-Lys568, Val566 | 1.6 ± 0.0 | 0.8 ± 0.3 |
| (pCABD2) (cl.1) | | |
| MG1655ΔcadAΔldc, P_{L-tac}adhE* (pCABD2) | 5.9 ± 0.2 | 1.8 ± 0.1 |

It can be seen from Table 3 that mutant alcohol dehydrogenases and a wild-type alcohol dehydrogenase was able to cause growth enhancement and accumulation of a higher amount of L-lysine as compared with WC196ΔcadAΔldc (pCABD2), in which neither expression of a wild-type nor a mutant alcohol dehydrogenase was increased.

### Example 12

### Construction of E. Coli MG1655ΔargR, P_{L-tac}adhE*

1. Construction of the strain MG1655ΔargR

This strain was constructed by inactivation of the native argR gene, which encodes a repressor of the L-arginine biosynthetic pathway in E. coli MG1655 by the kan gene. To replace the native argR gene, the DNA fragment carrying a kanamycin resistance marker (Km^{R}) encoded by the kan gene was integrated into the chromosome of E. coli MG1655 (ATCC 700926) in place of the native argR gene by the Red-driven integration.

A DNA fragment containing a Km^{R} marker encoded by the kan gene was obtained by PCR using the commercially available plasmid pACYC177 (GenBank/EMBL accession number X06402, “Fermentas”, Lithuania) as a template, and primers P26 (SEQ ID NO: 31) and P27 (SEQ ID NO: 32). Primer P26 contains 40 nucleotides homologous to the 5′-region of the argR gene introduced into the primer for further integration into the bacterial chromosome. Primer P27 contains 41 nucleotides homologous to the 3′-region of the argR gene introduced into the primer for further integration into the bacterial chromosome. The temperature profile was the following: initial DNA denaturation for 5 min at 95° C., followed by 25 cycles of denaturation at 95° C. for 30 sec, annealing at 55° C. for 30 sec, elongation at 72° C. for 40 sec; and the final elongation for 5 min at 72° C. Then, the amplified DNA fragment was purified by agarose gel-electrophoresis, extracted using “GenElute Spin Columns” (“Sigma”, USA) and precipitated by ethanol.

The obtained DNA fragment was used for electroporation and Red-mediated integration into the bacterial chromosome of the E. coli MG1655/pKD46.

MG1655/pKD46 cells were grown overnight at 30° C. in the liquid LB-medium with addition of ampicillin (100 μg/ml), then diluted 1:100 by the SOB-medium (Yeast extract, 5 g/l; NaCl, 0.5 g/l; Tryptone, 20 g/l; KCl, 2.5 mM; MgCl₂, 10 mM) with the addition of ampicillin (100 μg/ml) and L-arabinose (10 mM) (arabinose is used for inducing the plasmid encoding genes of Red system) and grown at 30° C. to reach the optical density of the bacterial culture OD₆₀₀=0.4-0.7. The grown cells from 10 ml of the bacterial culture were washed 3 times by the ice-cold de-ionized water, followed by suspending in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions. Shocked cells were added to 1-ml of SOC medium, incubated 2 hours at 37° C., and then were spread onto L-agar containing 25 μg/ml of chloramphenicol. Colonies grown within 24 h were tested for the presence of Km^{R} marker instead of the native argR gene by PCR using primers P28 (SEQ ID NO: 33) and P29 (SEQ ID NO: 34). For this purpose, a freshly isolated colony was suspended in 20 μl water and then 1 μl of obtained suspension was used for PCR. The temperature profile was the following: initial DNA denaturation for 5 min at 95° C.; then 30 cycles of denaturation at 95° C. for 30 sec, annealing at 55° C. for 30 sec and elongation at 72° C. for 30 sec; the final elongation for 5 min at 72° C. A few Km^{R} colonies tested contained the desired 1110 bp DNA fragment, confirming the presence of Km^{R} marker DNA instead of 660 bp fragment of argR gene. One of the obtained strains was cured from the thermosensitive plasmid pKD46 by culturing at 37° C. and the resulting strain was named E. coli MG1655ΔargR.

2. Construction of E. Coli MG1655ΔargR, P_{L-tac}adhE*.

E. coli MG1655ΔargR, P_{L-tac}adhE* was obtained by transduction of the P_{L-tac}, adhE* mutation from the strain MG1655::P_{L-tac}adhE*.

The strain MG1655ΔargR was infected with phage Plvir grown on the donor strain MG1655::P_{L-tac}adhE* and the strain MG1655ΔargR, P_{L-tac}adhE* was obtained. This strain was checked for growth on M9 plates with 2% ethanol as a sole carbon source. The growth rate was the same as for the strain MG1655::P_{L-tac}adhE* (36 h).

### Example 13

### Construction of the pMW119-ArgA4 Plasmid

ArgA gene with a single mutation provide the fbr (feedback resistant) phenotype (JP2002253268, EP1170361) and under the control of its own promoter was cloned into pMW119 vector.

The argA gene was obtained by PCR using the plasmid pKKArgA-r4 (JP2002253268, EP1170361) as a template, and primers P30 (SEQ ID NO: 35) and P31 (SEQ ID NO: 36). Sequence of the primer P30 homologous to the 5′-region of the argA gene located 20 bp upstream and 19 bp downstream of the start codon of the argA gene. Primer P31 contains 24 nucleotides homologous to the 3′-region of argA gene and HindIII restriction site introduced for further cloning into the pMW119/BamHI-HindIII vector.

Sequence of the P_{argA} promoter was obtained by PCR using E. coli MG1655 as a template, and primers P32 (SEQ ID NO: 37) and P33 (SEQ ID NO: 38). Primer P32 contains 30 nucleotides homologous to the 5′-untranslated region of the argA gene located 245 bp upstream of the start codon, and moreover this sequence includes BamHI recognition site. Primer P33 contains 24 nucleotides homologous to the 5′-region of the argA gene located 20 bp upstream of the start codon and start codon itself. The temperature profile was the following: initial DNA denaturation for 5 min at 95° C., followed by 25 cycles of denaturation at 95° C. for 30 sec, annealing at 54° C. for 30 sec, elongation at 72° C. for 1 min 20 sec (for ArgA gene) or 20 sec (for P_{argA} promoter) and the final elongation for 5 min at 72° C. Then, the amplified DNA fragments was purified by agarose gel-electrophoresis, extracted using “GenElute Spin Columns” (“Sigma”, USA) and precipitated by ethanol.

P_{argA}ArgA fragment was obtained by PCR using both the above-described DNA fragments: P_{argA} promoter and ArgA gene. First, the reaction mixture (total volume—100 μl) consisted of 10 μl of 10×PCR-buffer with 25 mM MgCl₂ (Sigma, USA), 200 μM each of dNTP and 1 U of Accu-Taq DNA polymerase (Sigma, USA). The argA fragment (25 ng) and P_{argA} (5 ng) were used as a template DNA and as primers simultaneously. Next, primers P31 and P32 were added in reaction mixture. The temperature profile was the following: 1st step-initial DNA denaturation for 5 min at 95° C., followed by 10 cycles of denaturation at 95° C. for 30 sec, annealing at 53° C. for 30 sec, elongation at 72° C. for 1 min, 2nd step—15 cycles of denaturation at 95° C., annealing at 54° C. for 30 sec, elongation at 72° C. for 1 min 30 sec. The amplified DNA fragments was purified by agarose gel-electrophoresis, extracted using “GenElute Spin Columns” (“Sigma”, USA), precipitated by ethanol, treated with BamHI and HindIII and ligated with pMW119/BamHI-HindIII vector. As a result the plasmid pMW119- ArgA4 was obtained.

### Example 14

### The Effect of Increasing adhE Gene Expression on L-Arginine Production

To evaluate the effect of enhancing expression of the mutant adhE gene on L-arginine production, E. coli strains MG1655ΔargR P_{L-tac}adhE* and MG1655ΔargR were each transformed by plasmid pMW119- ArgA4. 10 obtained colonies of each sort of transformants were cultivated at 37° C. for 18 hours in a nutrient broth supplemented with 150 mg/l of Ap and 0.1 ml of each of the obtained cultures was inoculated into 2 ml of fermentation medium in a 20×200 mm test tube and cultivated at 32° C. for 96 hours with a rotary shaker. After cultivation, the amount of L-arginine which accumulates in the medium was determined by paper chromatography using the following mobile phase: butanol: acetic acid:water=4:1:1 (v/v). A solution (2%) of ninhydrin in acetone was used as a visualizing reagent. A spot containing L-arginine was cut out, L-arginine was eluted in 0.5% water solution of CdCl₂, and the amount of L-arginine was estimated spectrophotometrically at 540 nm. The results of ten independent test tube fermentations are shown in Table 4. As follows from Table 4, MG1655ΔargR P_{L-tac}adhE* produced a higher amount of L-arginine, as compared with MG1655ΔargR P_{L-tac}adhE*, both in medium with supplemented glucose and without it.

The composition of the fermentation medium was as follows (g/l):

| | |
|---|---|
| Ethanol | 20 |
| Glucose | 0/5 |
| (NH₄)₂SO₄ | 25 |
| K₂HPO₄ | 2 |
| MgSO₄•7H₂O | 1.0 |
| Thiamine hydrochloride | 0.002 |
| Yeast extract | 5.0 |
| CaCO₃ | 33 |

MgSO₄.7H₂O, ethanol and CaCO₃ were each sterilized separately.

TABLE 4

| | | | Ethanol (2%) | |
|---|---|---|---|---|
| | Ethanol (2%) | | and glucose (5%) | |
| | | Amount of | | Amount of |
| | | L-arginine, | | L-arginine, |
| Strain | OD₅₅₀ | g/l | OD₅₅₀ | g/l |
| MG1655ΔargR | 1.3 ± 0.2 | <0.1 | 8.0 ± 0.4 | 1.5 ± 0.2 |
| (pMW-argAm4) | | | | |
| MG1655ΔargRcat- | 7.2 ± 0.4 | 0.8 ± 0.3 | 13.4 ± 0.3 | 1.9 ± 0.2 |
| P_{L-tac}-adhE* | | | | |
| (pMW-argAm4) | | | | |

### Example 15

### Construction of the L-Leucine Producing E. Coli Strain NS1391

The strain NS1391 was obtained as follows.

At first, a strain having inactivated acetolactate synthase genes (combination of ΔilvIH and ΔilvGM deletions) was constructed. The ilvIH genes (ΔilvIH::cat) were deleted from the wild-type strain E. coli K12 (VKPM B-7) by P1 transduction (Sambrook et al, “Molecular Cloning A Laboratory Manual, Second Edition”, Cold Spring Harbor Laboratory Press (1989). E. coli MG1655 ΔilvIH::cat was used as a donor strain. Deletion of the ilvIH operon in the strain MG1655 was conducted by means of the Red-driven integration. According to this procedure, the PCR primers P34 (SEQ ID NO: 39) and P35 (SEQ ID NO: 40) homologous to the both region adjacent to the ilvIH operon and gene conferring chloramphenicol resistance in the template plasmid were constructed. The plasmid pMW-attL-Cm-attR (PCT application WO 05/010175) was used as a template in a PCR reaction. Conditions for PCR were following: denaturation step for 3 min at 95° C.; profile for two first cycles: 1 min at 95° C., 30 sec at 34° C., 40 sec at 72° C.; profile for the last 30 cycles: 30 sec at 95° C., 30 sec at 50° C., 40 sec at 72° C.; final step: 5 min at 72° C. Obtained 1713 bp PCR product was purified in agarose gel and used for electroporation of E. coli MG1655/pKD46. Chloramphenicol resistant recombinants were selected after electroporation and verified by means of PCR with locus-specific primers P36 (SEQ ID NO: 41) and P37 (SEQ ID NO: 42). Conditions for PCR verification were the following: denaturation step for 3 min at 94° C.; profile for the 30 cycles: 30 sec at 94° C., 30 sec at 53° C., 1 min 20 sec at 72° C.; final step: 7 min at 72° C. PCR product, obtained in the reaction with the chromosomal DNA from parental IlvIH⁺ strain MG1655 as a template, was 2491 nt in length. PCR product, obtained in the reaction with the chromosomal DNA from mutant MG1655 ΔilvIH::cat strain as a template, was 1823 nt in length. As a result the strain MG1655 ΔilvIH::cat was obtained. After deletion of ilvIH genes (ΔilvIH::cat) from E. coli K12 (VKPM B-7) by P1 transduction, Cm^{R} transductants were selected. As a result the strain B-7 ΔilvIH::cat was obtained. To eliminate the chloramphenicol resistance marker from B-7 ΔilvIH::cat, cells were transformed with the plasmid pMW118-int-xis (Ap^{R}) (WO2005/010175). Ap^{R} clones were grown on LB agar plates containing 150 mg/l ampicillin at 30° C. Several tens of Ap^{R} clones were picked up and tested for chloramphenicol sensitivity. The plasmid pMW118-int-xis was eliminated from Cm^{S} cells by incubation on LB agar plates at 42° C. As a result, the strain B-7 ΔilvIH was obtained.

The ilvGM genes (ΔilvGM::cat) were deleted from E. coli B-7 ΔilvIH by P1 transduction. E. coli MG1655 ΔilvGM::cat was used as a donor strain. The ilvGM operon was deleted from the strain MG1655 by Red-driven integration. According to this procedure, the PCR primers P38 (SEQ ID NO: 43) and P39 (SEQ ID NO: 44) homologous to both the region adjacent to the ilvGM operon and the gene conferring chloramphenicol resistance in the template plasmid were constructed. The plasmid pMW-attL-Cm-attR (PCT application WO 05/010175) was used as a template in the PCR reaction. Conditions for PCR were the following: denaturation step for 3 min at 95° C.; profile for two first cycles: 1 min at 95° C., 30 sec at 34° C., 40 sec at 72° C.; profile for the last 30 cycles: 30 sec at 95° C., 30 sec at 50° C., 40 sec at 72° C.; final step: 5 min at 72° C.

The obtained 1713 bp PCR product was purified in agarose gel and used for electroporation of E. coli MG1655/pKD46. Chloramphenicol resistant recombinants were selected after electroporation and verified by means of PCR with locus-specific primers P40 (SEQ ID NO: 45) and P41 (SEQ ID NO: 46). Conditions for PCR verification were the following: denaturation step for 3 min at 94° C.; profile for the 30 cycles: 30 sec at 94° C., 30 sec at 54° C., 1 min 30 sec at 72° C.; final step: 7 min at 72° C. PCR product, obtained in the reaction with the chromosomal DNA from the parental strain MG1655 as a template, was 2209 nt in length. The PCR product, obtained in the reaction with the chromosomal DNA from mutant MG1655 ΔilvGM::cat strain as a template, was 1941 nt in length. As a result, the strain MG1655 ΔilvGM::cat was obtained. After deletion of ilvGM genes (ΔilvGM::cat) from E. coli B-7 ΔilvIH by P1 transduction, CmR transductants were selected. As a result the strain B-7 ΔilvIH ΔilvBN ΔilvGM::cat was obtained. The chloramphenicol resistance marker was eliminated from B-7 ΔilvIH ΔilvBN ΔilvGM::cat as described above. As a result, the strain B-7 ΔilvIH ΔilvGM was obtained.

The native regulator region of the ilvBN operon was replaced with the phage lambda P_{L} promoter by the Red-driven integration. For that purpose, the strain B7 ΔilvIH ΔilvGM with the sole AHAS I was used as an initial strain for such modification. According to the procedure of Red-driven integration, the PCR primers P42 (SEQ ID NO: 47) and P43 (SEQ ID NO:48) were constructed. Oligonucleotide P42 (SEQ ID NO: 47) was homologous to the region upstream of the ilvB gene and the region adjacent to the gene conferring antibiotic resistance which was present in the chromosomal DNA of BW25113 cat-P_{L}-yddG. Oligonucleotide P43 (SEQ ID NO: 48) was homologous to both the ilvB region and the region downstream from the P_{L} promoter which was present in the chromosome of BW25113 cat-P_{L}-yddG. Obtaining BW25113 cat-P_{L}-yddG has been described in detail previously (EP1449918A1, Russian patent RU2222596). The chromosomal DNA of strain BW25113 cat-P_{L}-yddG was used as a template for PCR. Conditions for PCR were the following: denaturation for 3 min at 95° C.; profile for two first cycles: 1 min at 95° C., 30 sec at 34° C., 40 sec at 72° C.; profile for the last 30 cycles: 30 sec at 95° C., 30 sec at 50° C., 40 sec at 72° C.; final step: 5 min at 72° C. As a result, the PCR product was obtained (SEQ ID NO: 49), purified in agarose gel, and used for electroporation of E. coli B-7 ΔilvIH ΔilvGM, which contains the plasmid pKD46 with temperature sensitive replication. Electrocompetent cells were prepared as follows: E. coli strain B-7 ΔilvIH ΔilvGM was grown overnight at 30° C. in LB medium containing ampicillin (100 mg/l), and the culture was diluted 100 times with 5 ml of SOB medium (Sambrook et al, “Molecular Cloning A Laboratory Manual, Second Edition”, Cold Spring Harbor Laboratory Press (1989)) with ampicillin and L-arabinose (1 mM). The cells were grown with aeration at 30° C. to an OD₆₀₀ of ≈0.6 and then made electrocompetent by concentrating 100-fold and washing three times with ice-cold deionized H₂O. Electroporation was performed using 70 μl of cells and ≈100 ng of PCR product. Following electroporation, the cells were incubated with 1 ml of SOC medium (Sambrook et al, “Molecular Cloning A Laboratory Manual, Second Edition”, Cold Spring Harbor Laboratory Press (1989)) at 37° C. for 2.5 h and after that plated onto L-agar and were grown at 37° C. to select Cm^{R} recombinants. Then, to eliminate the pKD46 plasmid, 2 passages on L-agar with Cm at 42° C. were performed and the obtained colonies were tested for sensitivity to ampicillin.

The obtained strain B7 ΔilvIH ΔilvGM cat-P_{L}-ilvBN was valine sensitive. New valine resistant spontaneous mutants of AHAS I were obtained from this strain. Strains which grew better on 1 g/l of valine were characterized.

Valine resistance mutations which were resistance to isoleucine were obtained, as well. Variants with a specific activity which was more than that of the wild-type were obtained. The nucleotide sequence of the mutant operons for mutant ilvBN4 was determined. It was revealed that IlvBN4 contained one point mutation in IlvN: N17K Asn-Lys (codon aac was replaced with aag). Obtained strain B7 ΔilvIH ΔilvGM cat-P_{L}-ilvBN4 was used for the following constructions.

Then, cat-P_{L}-ilvBN4 DNA fragment was transferred from E. coli B7 ΔilvIH ΔilvGM cat-P_{L}-ilvBN4 into E. coli MG1655 mini-Mu::scrKYABR (EP application 1149911) by P1 transduction. As a result the strain ESP214 was obtained. The chloramphenicol resistance marker was eliminated from the strain ESP214 as described above. As a result, the strain ESP215 was obtained.

Then the DNA fragment shown in (SEQ ID NO: 50) was used for electroporation of the strain ESP215/pKD46 for the purpose of subsequent integration into chromosome. This DNA fragment contained regions complementary to the 3′ region of the gene b1701 and to the 5′ region of the gene b1703 (these genes are adjacent to the gene pps), which are necessary for integration into the chromosome. It also contained an excisable chloramphenicol resistance marker cat, and a mutant ilvBN4 operon under the control of the constitutive promoter P_{L}. Electroporation was performed as described above. Selected Cm^{R} recombinants contained a deletion of the gene pps as a result of the integration of cat-P_{L}-ilvBN4 fragment into the chromosome. Thus the strain ESP216 was obtained. The chloramphenicol resistance marker was eliminated from the strain ESP216 as described above. As a result, the strain ESP217 was obtained.

At the next step, the mutant leuA gene (Gly479->Cys) under the control of the constitutive promoter P_{L} was introduced into the strain ESP217. The DNA fragment shown in (SEQ ID NO: 51) was used for electroporation of the strain ESP217/pKD46 for the purpose of subsequent integration into the chromosome. This DNA fragment contained the 35 nt-region, which is necessary for integration into the chromosome and homologous to the upstream region of the gene leuA. It also contained an excisable region complementary to the sequence of chloramphenicol resistance marker cat, and the mutant leuA (Gly479->Cys) gene under the control of the constitutive promoter P_{L}. Electroporation was performed as described above. Selected Cm^{R} recombinants contained the mutant gene leuA (Gly479->Cys) under the control of the constitutive promoter P_{L} integrated into the chromosome. Thus, the strain ESP220 was obtained. The chloramphenicol resistance marker was eliminated from the strain ESP220 as described above. As a result, the strain ESP221 was obtained.

Then, the DNA fragment shown in SEQ ID NO: 52 was used for electroporation of the strain ESP221/pKD46 for the purpose of subsequent integration into the chromosome. This DNA fragment contained the 35 nt-region homologous to the upstream region of the gene tyrB, which is necessary for integration into the chromosome. It also contained an excisable region complementary to the sequence of chloramphenicol resistance marker cat and the gene tyrB with a modified regulatory (−35) region. Electroporation was performed as described above. Selected Cm^{R} recombinants contained the gene tyrB with the modified regulatory (−35) region. Thus, the strain NS1390 was obtained. The chloramphenicol resistance marker was eliminated from the strain NS1390 as described above. As a result, the strain NS1391 was obtained. Leucine producing strain NS1391 was used for further work.

### Example 16

### The Effect of Increasing the Mutant adhE Gene Expression on L-Leucine Production

To test the effect of enhanced expression of the adhE gene under the control of a P_{L-tac} promoter on L-leucine production, DNA fragments from the chromosome of the above-described strain MG1655 P_{L-tac}adhE* were transferred to the L-leucine producing E. coli strain NS1391 by P1 transduction (Miller, J. H. (1972) Experiments in Molecular Genetics, Cold Spring Harbor Lab. Press, Plainview, N.Y.) to obtain the strain NS1391 P_{L-tac}adhE*.

Both E. coli strains, NS1391 and NS1391 P_{L-tac}adhE*, were cultured for 18-24 hours at 37° C. on L-agar plates. To obtain a seed culture, the strains were grown on a rotary shaker (250 rpm) at 32° C. for 18 hours in 20×200-mm test tubes containing 2 ml of L-broth supplemented with 4% sucrose. Then, the fermentation medium was inoculated with 0.21 ml of seed material (10%). The fermentation was performed in 2 ml of a minimal fermentation medium in 20×200-mm test tubes. Cells were grown for 48-72 hours at 32° C. with shaking at 250 rpm. The amount of L-leucine was measured by paper chromatography (liquid phase composition: butanol-acetic acid-water=4:1:1). The results of ten independent test tube fermentations are shown in Table 5. As follows from Table 5, NS1391 P_{L-tac}adhE* produced a higher amount of L-leucine, as compared with NS1391, in media containing different concentrations of ethanol.

The composition of the fermentation medium (g/l) (pH 7.2) was as follows:

| | |
|---|---|
| Glucose | 60.0 |
| Ethanol | 0/10.0/20.0/30.0 |
| (NH₄)₂SO₄ | 25.0 |
| K₂HPO₄ | 2.0 |
| MgSO₄•7H₂O | 1.0 |
| Thiamine | 0.01 |
| CaCO₃ | 25.0 |

Glucose, ethanol and CaCO₃ were sterilized separately.

TABLE 5

| | Glucose (6%) | | | | | | | |
|---|---|---|---|---|---|---|---|---|
| | without ethanol | | +1% ethanol | | +2% ethanol | | +3% ethanol | |
| Strain | Leu, g/l | OD₅₅₀ | Leu, g/l | OD₅₅₀ | Leu, g/l | OD₅₅₀ | Leu, g/l | OD₅₅₀ |
| NS1391 | 5.0 ± 0.1 | 34.2 ± 0.6 | 4.8 ± 0.1 | 31.7 ± 0.4 | 3.9 ± 0.2 | 31.3 ± 0.6 | 4.0 ± 0.1 | 28.0 ± 0.6 |
| NS1391 | 5.1 ± 0.1 | 31.1 ± 0.2 | 5.9 ± 0.1 | 29.3 ± 0.3 | 4.9 ± 0.1 | 27.3 ± 0.6 | 4.6 ± 0.1 | 22.8 ± 0.2 |
| P_{L-tac}-adhE* | | | | | | | | |

### Example 17

### The Effect of the Increasing the adhE Gene Expression on L-Phenylalanine Production

To test the effect of enhanced expression of the adhE gene under the control of a P_{L-tac} promoter on phenylalanine production, the DNA fragments from the chromosome of the above-described strains MG1655Δtdh, rhtA*, P_{L-tac}adhE; MG1655Δtdh, rhtA*, P_{L-tac}adhE*; MG1655Δtdh, rhtA*, P_{L-tac}adhE-Lys568 (cl. 18); MG1655Δtdh, rhtA*, P_{L-tac}adhE-Lys568, Val566 (cl. 1); MG1655Δtdh, rhtA*, adhE* can be transferred to the phenylalanine-producing E. coli strain AJ12739 by P1 transduction (Miller, J. H. (1972) Experiments in Molecular Genetics, Cold Spring Harbor Lab. Press, Plainview, N.Y.). The strain AJ12739 has been deposited in the Russian National Collection of Industrial Microorganisms (VKPM) (Russia, 117545 Moscow, 1 Dorozhny proezd, 1) on Nov. 6, 2001 under accession number VKPM B-8197 and then converted to a deposit under the Budapest Treaty on Aug. 23, 2002

The resulting strains and the parent strain AJ12739 can each be cultivated at 37° C. for 18 hours in a nutrient broth, and 0.3 ml of the obtained cultures can each be inoculated into 3 ml of a fermentation medium in a 20×200 mm test tube and cultivated at 37° C. for 48 hours with a rotary shaker. After cultivation, the amount of phenylalanine which accumulates in the medium can be determined by TLC. 10×15 cm TLC plates coated with 0.11 mm layers of Sorbfil silica gel without fluorescent indicator (Stock Company Sorbpolymer, Krasnodar, Russia) can be used. The Sorbfil plates can be developed with a mobile phase: propan-2-ol: ethylacetate: 25% aqueous ammonia: water=40:40:7:16 (v/v). A solution (2%) of ninhydrin in acetone can be used as a visualizing reagent.

The composition of the fermentation medium (g/l):

| | |
|---|---|
| Ethanol | 20.0 |
| (NH₄)₂SO₄ | 16.0 |
| K₂HPO₄ | 0.1 |
| MgSO₄•7H₂O | 1.0 |
| FeSO₄•7H₂O | 0.01 |
| MnSO₄•5H₂O | 0.01 |
| Thiamine HCl | 0.0002 |
| Yeast extract | 2.0 |
| Tyrosine | 0.125 |
| CaCO₃ | 20.0 |

Ethanol and magnesium sulfate are sterilized separately. CaCO₃ dry-heat sterilized at 180° C. for 2 hours. pH is adjusted to 7.0.

### Example 18

### The Effect of Increasing the adhE Gene Expression on L-Tryptophan Production

To test the effect of enhanced expression of the adhE gene under the control of a P_{L-tac} promoter on tryptophan production, the DNA fragments from the chromosome of the above-described strains MG1655Δtdh, rhtA*, P_{L-tac}adhE; MG1655Δtdh, rhtA*, P_{L-tac}adhE*; MG1655Δtdh, rhtA*, P_{L-tac}adhE-Lys568 (cl. 18); MG1655Δtdh, rhtA*, P_{L-tac}adhE-Lys568, Val566(cl. 1); MG1655Atdh, rhtA*, adhE* can be transferred to the tryptophan-producing E. coli strain SV164 (pGH5) by P1 transduction (Miller, J. H. (1972) Experiments in Molecular Genetics, Cold Spring Harbor Lab. Press, Plainview, N.Y.). The strain SV164 has the trpE allele encoding anthranilate synthase which is not subject to feedback inhibition by tryptophan. The plasmid pGH5 harbors a mutant serA gene encoding phosphoglycerate dehydrogenase which is not subject to feedback inhibition by serine. The strain SV164 (pGH5) is described in detail in U.S. Pat. No. 6,180,373 or European patent 0662143.

The resulting strains and the parent strain SV164 (pGH5) can each be cultivated with shaking at 37° C. for 18 hours in 3 ml of nutrient broth supplemented with 20 mg/l of tetracycline (marker of pGH5 plasmid). 0.3 ml of the obtained cultures can each be inoculated into 3 ml of a fermentation medium containing tetracycline (20 mg/l) in 20×200 mm test tubes, and cultivated at 37° C. for 48 hours with a rotary shaker at 250 rpm. After cultivation, the amount of tryptophan which accumulates in the medium can be determined by TLC as described in

### Example 17

The fermentation medium components are set forth in Table 6, but should be sterilized in separate groups A, B, C, D, E, F, and H, as shown, to avoid adverse interactions during sterilization.

TABLE 6

| Solutions | Component | Final concentration, g/l |
|---|---|---|
| A | KH₂PO₄ | 1.5 |
| | NaCl | 0.5 |
| | (NH₄)₂SO₄ | 1.5 |
| | L-Methionine | 0.05 |
| | L-Phenylalanine | 0.1 |
| | L-Tyrosine | 0.1 |
| | Mameno (total N) | 0.07 |
| B | Ethanol | 20.0 |
| | MgSO₄•7H₂O | 0.3 |
| C | CaCl₂ | 0.011 |
| D | FeSO₄•7H₂O | 0.075 |
| | Sodium citrate | 1.0 |
| E | Na₂MoO₄•2H₂O | 0.00015 |
| | H₃BO₃ | 0.0025 |
| | CoCl₂•6H₂O | 0.00007 |
| | CuSO₄•5H₂O | 0.00025 |
| | MnCl₂•4H₂O | 0.0016 |
| | ZnSO₄•7H₂O | 0.0003 |
| F | Thiamine HCl | 0.005 |
| G | CaCO₃ | 30.0 |
| H | Pyridoxine | 0.03 |

Solution A had a pH of 7.1, adjusted by NH₄OH.

### Example 19

### The Effect of the Increasing the adhE Gene Expression on L-Histidine Production

To test the effect of enhanced expression of the adhE gene under the control of a P_{L-tac} promoter on histidine production, the DNA fragments from the chromosome of the above-described strains MG1655Δtdh, rhtA*, P_{L-tac}adhE; MG1655Δtdh, rhtA*, P_{L-tac}adhE*; MG1655Δtdh, rhtA*, P_{L-tac}adhE-Lys568 (cl. 18); MG1655Δtdh, rhtA*, P_{L-tac}adhE-Lys568, Val566(cl. 1); MG1655Δtdh, rhtA*, adhE* can be transferred to the histidine-producing E. coli strain 80 by P1 transduction (Miller, J. H. (1972) Experiments in Molecular Genetics, Cold Spring Harbor Lab. Press, Plainview, N.Y.). The strain 80 has been described in Russian patent 2119536 and deposited in the Russian National Collection of Industrial Microorganisms (Russia, 117545 Moscow, 1 Dorozhny proezd, 1) on Oct. 15, 1999 under accession number VKPM B-7270 and then converted to a deposit under the Budapest Treaty on Jul. 12, 2004.

The resulting strains and the parent strain 80 can each be cultivated in L broth for 6 hours at 29° C. Then, 0.1 ml of obtained culture can each be inoculated into 2 ml of fermentation medium in a 20×200 mm test tube and cultivated for 65 hours at 29° C. with a rotary shaker (350 rpm). After cultivation, the amount of histidine which accumulates in the medium can be determined by paper chromatography. The paper can be developed with a mobile phase: n-butanol: acetic acid:water=4:1:1 (v/v). A solution of ninhydrin (0.5%) in acetone can be used as a visualizing reagent.

The composition of the fermentation medium (pH 6.0) (g/l):

| | |
|---|---|
| Ethanol | 20.0 |
| Mameno (soybean hydrolyzate) | 0.2 |
| | as total nitrogen |
| L-proline | 1.0 |
| (NH₄)₂SO₄ | 25.0 |
| KH₂PO₄ | 2.0 |
| MgSO₄•7H₂0 | 1.0 |
| FeSO₄•7H₂0 | 0.01 |
| MnSO₄ | 0.01 |
| Thiamine | 0.001 |
| Betaine | 2.0 |
| CaCO₃ | 60.0 |

Ethanol, proline, betaine and CaCO₃ are sterilized separately. pH is adjusted to 6.0 before sterilization.

### Example 20

### The Effect of Increasing the adhE Gene Expression on L-Glutamic Acid Production

To test the effect of enhanced expression of the adhE gene under the control of a P_{L-tac} promoter on glutamic acid production, the DNA fragments from the chromosome of the above-described strains MG1655Δtdh, rhtA*, P_{L-tac}adhE; MG1655Δtdh, rhtA*, P_{L-tac}adhE*; MG1655Δtdh, rhtA*, P_{L-tac}adhE-Lys568 (cl. 18); MG1655Δtdh, rhtA*, P_{L-tac}adhE-Lys568, Val566 (cl. 1); MG1655Δtdh, rhtA*, adhE* can be transferred to the glutamic acid-producing E. coli strain VL334thrC⁺ (EP1172433) by P1 transduction (Miller, J. H. (1972) Experiments in Molecular Genetics, Cold Spring Harbor Lab. Press, Plainview, N.Y.). The strain VL334thrC⁺ has been deposited in the Russian National Collection of Industrial Microorganisms (VKPM) (Russia, 117545 Moscow, 1 Dorozhny proezd, 1) on Dec. 6, 2004 under the accession number VKPM B-8961 and then converted to a deposit under the Budapest Treaty on Dec. 8, 2004.

The resulting strains and the parent strain VL334thrC⁺ can each be cultivated with shaking at 37° C. for 18 hours in 3 ml of nutrient broth. 0.3 ml of the obtained cultures can each be inoculated into 3 ml of a fermentation medium in 20×200 mm test tubes, and cultivated at 37° C. for 48 hours with a rotary shaker at 250 rpm.

The composition of the fermentation medium (pH 7.2) (g/l):

| | |
|---|---|
| Ethanol | 20.0 |
| Ammonium sulfate | 25.0 |
| KH₂PO₄ | 2.0 |
| MgSO₄•7H₂O | 1.0 |
| Thiamine | 0.0001 |
| L-isoleucine | 0.05 |
| CaCO₃ | 25.0 |

Ethanol and CaCO₃ were sterilized separately.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

### INDUSTRIAL APPLICABILITY

According to the present invention, production of an L-amino acid by a bacterium of the Enterobacteriaceae family can be enhanced.

1. A method for producing an L-amino acid comprising:
A) cultivating in a culture medium containing ethanol an L-amino acid-producing bacterium of the Enterobacteriaceae family having an alcohol dehydrogenase, and
B) isolating the L-amino acid from the culture medium,
wherein the gene encoding said alcohol dehydrogenase is expressed under the control of a non-native promoter which functions under aerobic cultivation conditions.

2. The method according to claim 1, wherein said non-native promoter is selected from the group consisting of P_{tac}, P_{lac}, Pₜᵣₚ, P_{trc}, P_{R}, and P_{L}.

3. The method according to claim 1, wherein said alcohol dehydrogenase is resistant to aerobic inactivation.

4. The method according to claim 1, wherein said alcohol dehydrogenase originates from a bacterium selected from the group consisting of Escherichia coli, Erwinia carotovora, Salmonella typhimurium, Shigella flexneri, Yersinia pestis, Pantoea ananatis, Lactobacillus plantarum, and Lactococcus lactis.

5. The method according to claim 1, wherein said alcohol dehydrogenase comprises the amino acid sequence set forth in SEQ ID NO: 2, except the glutamic acid residue at position 568 is replaced with another amino acid residue other than an aspartic acid residue.

6. The method according to claim 1, wherein said alcohol dehydrogenase comprises the amino acid sequence set forth in SEQ ID NO: 2, except the glutamic acid residue at position 568 is replaced with a lysine residue.

7. The method according to claim 5, wherein said alcohol dehydrogenase has at least one additional mutation which is able to improve the growth of said bacterium in a liquid medium which contains ethanol as the sole carbon source.

8. The method according to claim 7, wherein said additional mutation is selected from the group consisting of:
A) replacement of the glutamic acid residue at position 560 in SEQ ID NO: 2 with another amino acid residue;
B) replacement of the phenylalanine residue at position 566 in SEQ ID NO: 2 with another amino acid residue;
C) replacement of the glutamic acid residue, the methionine residue, the tyrosine residue, the isoleucine residue and the alanine residue at positions 22, 236, 461, 554, and 786, respectively, in SEQ ID NO: 2 with other amino acid residues; and
D) combinations thereof.

9. The method according to claim 7, wherein said additional mutation is selected from the group consisting of:
A) replacement of the glutamic acid residue at position 560 in SEQ ID NO: 2 with a lysine residue;
B) replacement of the phenylalanine residue at position 566 in SEQ ID NO: 2 with a valine residue;
C) replacement of the glutamic acid residue, the methionine residue, the tyrosine residue, the isoleucine residue and the alanine residue at positions 22, 236, 461, 554, and 786, respectively, in SEQ ID NO: 2 with a glycine residue, a valine residue, a cysteine residue, a serine residue, and a valine residue, respectively; and
D) combinations thereof.

10. The method according to claim 1, wherein said L-amino acid-producing bacterium belongs to a genus selected from the group consisting of Escherichia, Enterobacter, Erwinia, Klebsiella, Pantoea, Providencia, Salmonella, Serratia, Shigella, and Morganella.

11. The method according to claim 1, wherein said L-amino acid is selected from the group consisting of L-threonine, L-lysine, L-histidine, L-phenylalanine, L-arginine, L-tryptophan, L-glutamic acid, and L-leucine.


SEQUENCE LISTING (58 sequences)

SEQ ID NO: 1 - DNA, 2676 bp, Escherichia coli
  CDS (1)..(2676)
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 2 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 3 - DNA, 70 bp, Artificial
  primer P1
gatgaaagcgttatccaaactgaaagcggaagaggccgacgcactttgcgccgaataaatacctgtgacg

SEQ ID NO: 4 - DNA, 69 bp, Artificial
  primer P2
ttaatcccagctcagaataactttcccggactttacgccccgccctgccactcatcgcagtactgttgt

SEQ ID NO: 5 - DNA, 18 bp, Artificial
  primer P3
cggtcatgcttggtgatg

SEQ ID NO: 6 - DNA, 21 bp, Artificial
  primer P4
ttaatcccagctcagaataac

SEQ ID NO: 7 - DNA, 183 bp, Artificial
  hybrid promoter
ctagatctctcacctaccaaacaatgcccccctgcaaaaaataaattcataaaaaacatacagataaccatctgcggtgataaattatctctggcggtgttgacaattaatcatcggctcgtataatgtgtggaattgtgagcggtttaacattatcaggagagcattatggctgttactaat

SEQ ID NO: 8 - DNA, 69 bp, Artificial
  primer P5
cgttattgttatctagttgtgcaaaacatgctaatgtagcattacgccccgccctgccactcatcgcag

SEQ ID NO: 9 - DNA, 58 bp, Artificial
  primer P6
attagtaacagccataatgctctcctgataatgttaaaccgctcacaattccacacat

SEQ ID NO: 10 - DNA, 24 bp, Artificial
  primer P7
acttgttcttgagtgaaactggca

SEQ ID NO: 11 - DNA, 22 bp, Artificial
  primer P8
aagacgcgctgacaatacgcct

SEQ ID NO: 12 - DNA, 69 bp, Artificial
  primer P9
cgttattgttatctagttgtgcaaaacatgctaatgtagcatcagaaaaactcatcgagcatcaaatga

SEQ ID NO: 13 - DNA, 65 bp, Artificial
  primer P10
agccggagcagcttctttcttcgctgcagtttcaccttctacgttgtgtctcaaaatctctgatg

SEQ ID NO: 14 - DNA, 24 bp, Artificial
  primer P11
aagacgcgctgacaatacgccttt

SEQ ID NO: 15 - DNA, 24 bp, Artificial
  primer P12
aaggggccgtttatgttgccagac

SEQ ID NO: 16 - DNA, 69 bp, Artificial
  primer P13
catgtgggttatgtacgaacatccggaaactcacttcgaaaagtcggcgctgcgctttatggatatccg

SEQ ID NO: 17 - DNA, 69 bp, Artificial
  primer P14
atggacgccgcgaagatcatgtgggttatgtacgaacatcccgttgtgtctcaaaatctctgatgttac

SEQ ID NO: 18 - DNA, 67 bp, Artificial
  primer P15
cattttcgggaacttgtagatacgtttacggatatccattcagaaaaactcatcgagcatcaaatga

SEQ ID NO: 19 - DNA, 23 bp, Artificial
  primer P16
cttcgaagtagaagcggacccga

SEQ ID NO: 20 - DNA, 27 bp, Artificial
  primer P17
ccagaagtggtggtgacagcgatcatt

SEQ ID NO: 21 - DNA, 75 bp, Artificial
  primer P18
atggacgccgcgaagatcatgtgggttatgtacgaacatccggaaactcacttcgaaaagctggcgctgcgcttt

SEQ ID NO: 22 - DNA, 73 bp, Artificial
  primer P19
cattttcgggaacttgtagatacgtttacggatatccataaagcgcagcgccagcttttcgaagtgagtttcc

SEQ ID NO: 23 - DNA, 31 bp, Artificial
  primer P20
atcgaattcaagacgcgctgacaatacgcct

SEQ ID NO: 24 - DNA, 27 bp, Artificial
  primer P21
cacgctctacgagtgcgttaagttcag

SEQ ID NO: 25 - DNA, 69 bp, Artificial
  primer P22
aagcaaaagccggataatgttagccataaataaggttgaaatcagaaaaactcatcgagcatcaaatga

SEQ ID NO: 26 - DNA, 30 bp, Artificial
  primer P23
ttcgaattcgttgtgtctcaaaatctccga

SEQ ID NO: 27 - DNA, 21 bp, Artificial
  primer P24
cgtcttcagacagaacaccac

SEQ ID NO: 28 - DNA, 23 bp, Artificial
  primer P25
atgcttgatggtcggaagaggca

SEQ ID NO: 29 - DNA, 2688 bp, Pantoea ananatis
  CDS (1)..(2688)
atggccgttactaatgtcgctgaactcaatgcactggttgaacgtgtaaaaaaagcccagcaagaattcgccaatttttctcaacaacaggtcgatgccatcttccgcgcagccgcactggccgccgcggatgcccgaattccactcgctaaaatggcggtggcggaatcgggcatgggcattgttgaagacaaagtcattaaaaatcacttcgcttctgaatacatctacaacgcctataaggatgagaaaacctgcggcgtactggacaccgatgatacgtttggcaccatcaccatcgctgaacccatcggcctgatttgcggtattgtccccaccactaaccctacctcgaccgcaatttttaaggcacttatcagccttaaaacccgcaacgggattatcttctccccccatcctcgagccaaagatgcgacgaacaaagcggcggatattgtcctgcaggcagcgattgccgctggcgcgcccaaagacattataggctggattgatgcaccttctgtggaactgtccaatcagttgatgcaccatcctgatattaacctgattctggcgacgggtggccccggcatggtcaaagccgcctacagctcaggtaagccggcgattggcgtgggggccggtaacacgcccgttgtcatcgatgaaacagctgatgttaaacgcgccgttgcctccatcctgatgtcaaaaacgtttgataacggtgtgatctgtgcctctgaacagtcggttatcgtggtggatgccgtctacgacgccgtgcgcgagcgcttcgccagccatggtggctatttgcttcagggacaggagctgagtgcggtacaaaatatcattctaaaaaacggtgggcttaacgccgccattgtgggccagcctgcggtgaagattgcggagatggccggcatcagcgtacctggtgaaaccaaaatcctgattggcgaagttgaacgggtcgatgaatcagaacctttcgctcatgaaaaactgtcgccgacactggcgatgtaccgtgctaaagattatcaggatgccgtcagcaaagcggagaaactggtggcgatgggcggtattggtcatacgtcatgcctgtataccgaccaggacaatcagacagcgcgcgtgcactattttggcgacaagatgaaaacagcccgcattctgatcaacacgccagcttctcagggcggtattggtgatttatataacttcaaactcgccccttctctgacactgggttgtggttcctggggcggtaactccatttctgaaaacgtggggcccaaacatctcatcaacaagaaaaccgtcgctaagcgagctgaaaatatgttgtggcataaacttccgaagtccatttactttcgtcgcggctctttacccattgcgcttgaagagatcgccactgacggtgccaaacgcgcgtttgtggtgactgaccgcttcctgtttaacaacggttatgccgatcaggtcacccgcgttttaaaatctcacggcatcgaaaccgaagttttctttgaggttgaagcggatcccaccttaagcatcgtgcgtaaaggtgcagaacagatgaacagctttaagccagacgtgatcatcgccctgggcggcggttcgccgatggatgcagccaaaatcatgtgggtcatgtatgagcatccagaaacccattttgaagagctggcactgcggtttatggatattcgcaaacgtatctataagttccctaaaatgggcgtgaaagcgcgcatggtggccattacgacaacctcaggcacaggttcagaagtgacgccttttgccgtggtaacggatgacgcgaccggacagaaatacccgctggccgattatgcgctgacgccggatatggctatcgttgatgccaacctggtcatggatatgccacgttcactttgtgccttcggcggtctggatgcggtgacgcacgcgctggaagcctatgtgtccgttctggccaatgaatactccgatggtcaggccctgcaggcgcttaagctgcttaaagagaacttaccggcgagttatgcagaaggtgcaaaaaatccggttgcccgtgaacgtgtacataatgccgccaccatcgccggtatcgcctttgctaacgccttcctcggggtttgtcactcaatggcgcataagcttggctctgagttccatattcctcatggactggctaactcgctgctgatttccaacgttattcgctataacgccaatgacaaccctactaagcaaaccgcattcagccagtacgatcgtccccaggcgcgtcgtcgttatgctgaaattgcggatcatcttggtctcaccgcgacgggcgaccgcactgcccagaaaattgagaagctgctggtatggctggatgagatcaaaacggaactgggtattccggcatcgattcgtgaagccggtgtgcaggaggcagacttcctggcgaaagtcgataaactggcggatgatgcctttgatgaccagtgtactggcgcgaatccacgttatccgctgattgccgaactcaaacagctgatgctggacagctactacggacgcaaatttgtcgagccgttcgccagtgccgccgaggctgcccaggctcagcctgtcagtgacagcaaagcggcgaagaaagctaaaaaagcctag

SEQ ID NO: 30 - protein, 895 aa, Pantoea ananatis
MAVTNVAELNALVERVKKAQQEFANFSQQQVDAIFRAAALAAADARIPLAKMAVAESGMGIVEDKVIKNHFASEYIYNAYKDEKTCGVLDTDDTFGTITIAEPIGLICGIVPTTNPTSTAIFKALISLKTRNGIIFSPHPRAKDATNKAADIVLQAAIAAGAPKDIIGWIDAPSVELSNQLMHHPDINLILATGGPGMVKAAYSSGKPAIGVGAGNTPVVIDETADVKRAVASILMSKTFDNGVICASEQSVIVVDAVYDAVRERFASHGGYLLQGQELSAVQNIILKNGGLNAAIVGQPAVKIAEMAGISVPGETKILIGEVERVDESEPFAHEKLSPTLAMYRAKDYQDAVSKAEKLVAMGGIGHTSCLYTDQDNQTARVHYFGDKMKTARILINTPASQGGIGDLYNFKLAPSLTLGCGSWGGNSISENVGPKHLINKKTVAKRAENMLWHKLPKSIYFRRGSLPIALEEIATDGAKRAFVVTDRFLFNNGYADQVTRVLKSHGIETEVFFEVEADPTLSIVRKGAEQMNSFKPDVIIALGGGSPMDAAKIMWVMYEHPETHFEELALRFMDIRKRIYKFPKMGVKARMVAITTTSGTGSEVTPFAVVTDDATGQKYPLADYALTPDMAIVDANLVMDMPRSLCAFGGLDAVTHALEAYVSVLANEYSDGQALQALKLLKENLPASYAEGAKNPVARERVHNAATIAGIAFANAFLGVCHSMAHKLGSEFHIPHGLANSLLISNVIRYNANDNPTKQTAFSQYDRPQARRRYAEIADHLGLTATGDRTAQKIEKLLVWLDEIKTELGIPASIREAGVQEADFLAKVDKLADDAFDDQCTGANPRYPLIAELKQLMLDSYYGRKFVEPFASAAEAAQAQPVSDSKAAKKAKKA

SEQ ID NO: 31 - DNA, 69 bp, Artificial
  primer P26
tgcacaataatgttgtatcaaccaccatatcgggtgacttatcagaaaaactcatcgagcatcaaatga

SEQ ID NO: 32 - DNA, 64 bp, Artificial
  primer P27
gcaacattttccccgccgtcagaaacgacggggcagagattcgttgtgtctcaaaatctcgtat

SEQ ID NO: 33 - DNA, 24 bp, Artificial
  primer P28
aatcccgctctttcataacattat

SEQ ID NO: 34 - DNA, 23 bp, Artificial
  primer P29
attaatcgcaggggaaagcaggg

SEQ ID NO: 35 - DNA, 42 bp, Artificial
  primer P30
atcatgcaaagaggtgtgccgtggtaaaggaacgtaaaaccg

SEQ ID NO: 36 - DNA, 42 bp, Artificial; identical to SEQ ID NO: 35
  primer P31

SEQ ID NO: 37 - DNA, 30 bp, Artificial
  primer P32
gttggatcctgacatgcctctcccgagcaa

SEQ ID NO: 38 - DNA, 24 bp, Artificial
  primer P33
ccacggcacacctctttgcatgat

SEQ ID NO: 39 - DNA, 61 bp, Artificial
  primer P34
ttcacctttcctcctgtttattcttattacccctgaagcctgcttttttatactaagttgg

SEQ ID NO: 40 - DNA, 64 bp, Artificial
  primer P35
acatgttgggctgtaaattgcgcattgagatcattccgctcaagttagtataaaaaagctgaac

SEQ ID NO: 41 - DNA, 21 bp, Artificial
  primer P36
ttgctgtaagttgtgggattc

SEQ ID NO: 42 - DNA, 20 bp, Artificial
  primer P37
tccaggttcccactgatttc

SEQ ID NO: 43 - DNA, 64 bp, Artificial
  primer P38
gtttctcaagattcaggacggggaactaactatgaatgaagcctgcttttttatactaagttgg

SEQ ID NO: 44 - DNA, 64 bp, Artificial
  primer P39
tcagctttcttcgtggtcatttttatattccttttgcgctcaagttagtataaaaaagctgaac

SEQ ID NO: 45 - DNA, 20 bp, Artificial
  primer P40
tggtcgtgattagcgtggtg

SEQ ID NO: 46 - DNA, 20 bp, Artificial
  primer P41
cacatgcaccttcgcgtctt

SEQ ID NO: 47 - DNA, 64 bp, Artificial
  primer P42
ccgcaggcgactgacgaaacctcgctccggcggggtcgctcaagttagtataaaaaagctgaac

SEQ ID NO: 48 - DNA, 64 bp, Artificial
  primer P43
tgcccgaacttgccatgctccagtctccttcttctgagctgtttccttctagacggccaatgct

SEQ ID NO: 49 - DNA, 1968 bp, artificial
  DNA fragment containing cat gene and PL promoter
ccgcaggcgactgacgaaacctcgctccggcggggtcgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccgatatctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcaagatcttcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcagcattcaaagcagaaggctttggggtgtgtgatacgaaacgaagcattggccgtctagaaggaaacagctcagaagaaggagactggagcatggcaagttcgggca

SEQ ID NO: 50 - DNA, 4971 bp, DNA fragment
aatctgatccttcactgcccgtgcacggctctcattttcgacaaacggcagcgtgatgctgctgatagtgacgggaaattgtgacattactgcgtgtcctaatacctccgcagttattgccgtaccatcagaaatataaaaaacgtggcgatcaacagcattatccattttgttcttcccgtgatgcagacataattctatgaaagcataaattaaaaacgcacagaagcgtagaacgttatgtctggtttataaaatgaaccttcaattttattttttatgaaaacagcatttcatttttatggtttcgtttataccgatggtttatgtggaaattgtcgaagagagcagatttgcgcaacgctgggatcagtcttaaaaagtaaaaaaatatatttgcttgaacgattcaccgtttttttcatccggttaaatatgcaaagataaatgcgcagaaatgtgtttctcaaaccgttcatttatcacaaaaggattgttcgatgtccaacaatggctcgtaaccgctggtgctttggtcgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccgatatctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcaagatcttcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcagcattcaaagcagaaggctttggggtgtgtgatacgaaacgaagcattggccgtctagaaggaaacagctcagaagaaggagactggagcatggcaagttcgggcacaacatcgacgcgtaagcgctttaccggcgcagaatttatcgttcatttcctggaacagcagggcattaagattgtgacaggcattccgggcggttctatcctgcctgtttacgatgccttaagccaaagcacgcaaatccgccatattctggcccgtcatgaacagggcgcgggctttatcgctcagggaatggcgcgcaccgacggtaaaccggcggtctgtatggcctgtagcggaccgggtgcgactaacctggtgaccgccattgccgatgcgcggctggactccatcccgctgatttgcatcactggtcaggttcccgcctcgatgatcggcaccgacgccttccaggaagtggacacctacggcatctctatccccatcaccaaacacaactatctggtcagacatatcgaagaactcccgcaggtcatgagcgatgccttccgcattgcgcaatcaggccgcccaggcccggtgtggatagacattcctaaggatgtgcaaacggcagtttttgagattgaaacacagcccgctatggcagaaaaagccgccgcccccgcctttagcgaagaaagcattcgtgacgcagcggcgatgattaacgctgccaaacgcccggtgctttatctgggcggcggtgtgatcaatgcgcccgcacgggtgcgtgaactggcggagaaagcgcaactgcctaccaccatgactttaatggcgctgggcatgttgccaaaagcgcatccgttgtcgctgggtatgctggggatgcacggcgtgcgcagcaccaactatattttgcaggaggcggatttgttgatagtgctcggtgcgcgttttgatgaccgggcgattggcaaaaccgagcagttctgtccgaatgccaaaatcattcatgtcgatatcgaccgtgcagagctgggtaaaatcaagcagccgcacgtggcgattcaggcggatgttgatgacgtgctggcgcagttgatcccgctggtggaagcgcaaccgcgtgcagagtggcaccagttggtagcggatttgcagcgtgagtttccgtgtccaatcccgaaagcgtgcgatccgttaagccattacggcctgatcaacgccgttgccgcctgtgtcgatgacaatgcaattatcaccaccgacgttggtcagcatcagatgtggaccgcgcaagcttatccgctcaatcgcccacgccagtggctgacctccggtgggctgggcacgatgggttttggcctgcctgcggcgattggcgctgcgctggcgaacccggatcgcaaagtgttgtgtttctccggcgacggcagcctgatgatgaatattcaggagatggcgaccgccagtgaaaatcagctggatgtcaaaatcattctgatgaacaacgaagcgctggggctggtgcatcagcaacagagtctgttctacgagcaaggcgtttttgccgccacctatccgggcaaaatcaactttatgcagattgccgccggattcggcctcgaaacctgtgatttgaataacgaagccgatccgcaggcttcattgcaggaaatcatcaatcgccctggcccggcgctgatccatgtgcgcattgatgccgaagaaaaagtttacccgatggtgccgccaggtgcggcgaatactgaaatggtgggggaataagccatgcaaaacacaactcatgacaacgtaattctggagctcaccgttcgcaaccatccgggcgtaatgacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaacaaacctggttaagcctggctgaactgaagaaataaaataaatccccggcggcgtttagtcgccggggttatgtgatccccgaagatgaaacttattcaatctcttcacagacatcctgcgttaaacgccgcataatatcttttcttaacaaaaacttttgtattttacctgaggtagttcgcggtagtttttcgattaccacgatatgttcaggatatttatattttgcgacccgtttacggctaaaaaaagccactacctcttccagcgataatgaatgatgcggcgctttcagcacgacataagcgcatgatcgttcacctaaacgttcatcggacattgcaaccacacaggcatcgtgaattttaggatgctgcaataaaatatcttccacttcacggctgctaatattttcgccgccgcggacaataatatcttttttgcgtccggtaatttttatatagccagcctcatccatacggcagagatcgccgctgtaataccagccttcttcatccagggcacgggcggtta

SEQ ID NO: 51 - DNA, 3786 bp, DNA fragment
aaatgtgcttatttaataattaatttatatatttaatgcattaattcttaacattaattgatcaataatattcaccaaatcaatatcaaaaaaaatcgcaaaacatataattcaatacaaatcatcaggataggttttgcaacgcgtgcattttgtcccctttttcctcgttgattagatgcaaaaatttatgctgaaatatgtcaaccgatgaaaagcgtcggtagttaagcagaaattaatatcgcttactttaaccaccgcagcacaattagctaattttacggatgcagaactcacgctggcgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccgatatctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcaagatcttcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcagcattcaaagcagaaggctttggggtgtgtgatacgaaacgaagcattggccgtctagaaggaaacagctaaggacccaaaccatgagccagcaagtcattattttcgataccacattgcgcgacggtgaacaggcgttacaggcaagcttgagtgtgaaagaaaaactgcaaattgcgctggcccttgagcgtatgggtgttgacgtgatggaagtcggtttccccgtctcttcgccgggcgattttgaatcggtgcaaaccatcgcccgccaggttaaaaacagccgcgtatgtgcgttagctcgctgcgtggaaaaagatatcgacgtggcggccgaatccctgaaagtcgccgaagccttccgtattcatacctttattgccacttcgccaatgcacatcgccaccaagctgcgcagcacgctggacgaggtgatcgaacgcgctatctatatggtgaaacgcgcccgtaattacaccgatgatgttgaattttcttgcgaagatgccgggcgtacacccattgccgatctggcgcgagtggtcgaagcggcgattaatgccggtgccaccaccatcaacattccggacaccgtgggctacaccatgccgtttgagttcgccggaatcatcagcggcctgtatgaacgcgtgcctaacatcgacaaagccattatctccgtacatacccacgacgatttgggcctggcggtcggaaactcactggcggcggtacatgccggtgcacgccaggtggaaggcgcaatgaacgggatcggcgagcgtgccggaaactgttccctggaagaagtcatcatggcgatcaaagttcgtaaggatattctcaacgtccacaccgccattaatcaccaggagatatggcgcaccagccagttagttagccagatttgtaatatgccgatcccggcaaacaaagccattgttggcagcggcgcattcgcacactcctccggtatacaccaggatggcgtgctgaaaaaccgcgaaaactacgaaatcatgacaccagaatctattggtctgaaccaaatccagctgaatctgacctctcgttcggggcgtgcggcggtgaaacatcgcatggatgagatggggtataaagaaagtgaatataatttagacaatttgtacgatgctttcctgaagctggcggacaaaaaaggtcaggtgtttgattacgatctggaggcgctggccttcatcggtaagcagcaagaagagccggagcatttccgtctggattacttcagcgtgcagtctggctctaacgatatcgccaccgccgccgtcaaactggcctgtggcgaagaagtcaaagcagaagccgccaacggtaacggtccggtcgatgccgtctatcaggcaattaaccgcatcactgaatataacgtcgaactggtgaaatacagcctgaccgccaaaggccacggtaaagatgcgctgggtcaggtggatatcgtcgctaactacaacggtcgccgcttccacggcgtctgcctggctaccgatattgtcgagtcatctgccaaagccatggtgcacgttctgaacaatatctggcgtgccgcagaagtcgaaaaagagttgcaacgcaaagctcaacacaacgaaaacaacaaggaaaccgtgtga

SEQ ID NO: 52 - DNA, 2942 bp, DNA fragment
gtgcctctggcggatgtacgtttgtcatgagtctcacgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccgatatctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcatagacattcgccttcttccggtttattatgttttaaccacctgcccgtaaacctggagaaccatcgctctagaaggaaacagctatgtttcaaaaagttgacgcctacgctggcgacccgattcttacgcttatggagcgttttaaagaagaccctcgcagcgacaaagtgaatttaagtatcggtctgtactacaacgaagacggaattattccacaactgcaagccgtggcggaggcggaagcgcgcctgaatgcgcagcctcatggcgcttcgctttatttaccgatggaagggcttaactgctatcgccatgccattgcgccgctgctgtttggtgcggaccatccggtactgaaacaacagcgcgtagcaaccattcaaacccttggcggctccggggcattgaaagtgggcgcggatttcctgaaacgctacttcccggaatcaggcgtctgggtcagcgatcctacctgggaaaaccacgtagcaatattcgccggggctggattcgaagtgagtacttacccctggtatgacgaagcgactaacggcgtgcgctttaatgacctgttggcgacgctgaaaacattacctgcccgcagtattgtgttgctgcatccatgttgccacaacccaacgggtgccgatctcactaatgatcagtgggatgcggtgattgaaattctcaaagcccgcgagcttattccattcctcgatattgcctatcaaggatttggtgccggtatggaagaggatgcctacgctattcgcgccattgccagcgctggattacccgctctggtgagcaattcgttctcgaaaattttctccctttacggcgagcgcgtcggcggactttctgttatgtgtgaagatgccgaagccgctggccgcgtactggggcaattgaaagcaacagttcgccgcaactactccagcccgccgaattttggtgcgcaggtggtggctgcagtgctgaatgacgaggcattgaaagccagctggctggcggaagtagaagagatgcgtactcgcattctggcaatgcgtcaggaattggtgaaggtattaagcacagagatgccagaacgcaatttcgattatctgcttaatcagcgcggcatgttcagttataccggtttaagtgccgctcaggttgaccgactacgtgaagaatttggtgtctatctcatcgccagcggtcgcatgtgtgtcgccgggttaaatacggcaaatgtacaacgtgtggcaaaggcgtttgctgcggtgatgtaa

SEQ ID NO: 53 - protein, 891 aa, Shigella flexneri
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEDETAAKKEAAPAKAEKKAKKSA

SEQ ID NO: 54 - protein, 891 aa, Yersinia pestis
MAVTNVAELNELVARVKKAQREYANFSQEQVDKIFRAAALAAADARIPLAKLAVTESGMGIVEDKVIKNHFASEYIYNAYKDEKTCGILCEDKTFGTITIAEPIGLICGIVPTTNPTSTAIFKALISLKTRNGIIFSPHPRAKDATNKAADIVLQAAIAAGAPADIIGWIDAPTVELSNQLMHHPDINLILATGGPGMVKAAYSSGKPAIGVGAGNTPVVVDETADIKRVVASILMSKTFDNGVICASEQSIIVVDSVYDAVRERFASHGGYLLQGKELKAVQDIILKNGGLNAAIVGQPATKIAEMAGIKVPSNTKILIGEVKVVDESEPFAHEKLSPTLAMYRAKNFEEAVEKAEKLVEMGGIGHTSCLYTDQDNQTARVKYFGDKMKTARILINTPASQGGIGDLYNFKLAPSLTLGCGSWGGNSISENVGPKHLINKKTVAKRAENMLWHKLPKSIYFRRGSLPIALEEVATDGAKRAFIVTDRYLFNNGYADQVTSVLKSHGIETEVFFEVEADPTLSIVRKGAEQMNSFKPDVIIALGGGSPMDAAKIMWVMYEHPETHFEELALRFMDIRKRIYKFPKMGVKAKLVAITTTSGTGSEVTPFAVVTDDATGQKYPLADYALTPDMAIVDANLVMNMPKSLCAFGGLDAVTHALEAYVSVLANEYSDGQALQALKLLKEFLPASYNEGAKNPVARERVHNAATIAGIAFANAFLGVCHSMAHKLGSEFHIPHGLANAMLISNVIRYNANDNPTKQTAFSQYDRPQARRRYAEIADHLGLSAPGDRTAQKIQKLLAWLDEIKAELGIPASIREAGVQEADFLAKVDKLSEDAFDDQCTGANPRYPLISELKQILMDTYYGREYVEEFDREEEVAAATAPKAEKKTKK

SEQ ID NO: 55 - protein, 891 aa, Erwinia carotovora
MAVTNVAELNALVERVKKAQQEFATYTQEQVDKIFRAAALAASDARIPLAKMAVAESGMGIVEDKVIKNHFASEYIYNAYQDEKTCGVLSTDDTFGTITIAEPIGLICGIVPTTNPTSTAIFKALISLKTRNGIIFSPHPRAKNATNKAADIVLQAAIAAGAPKDIIGWIDQPSVDLSNQLMHHPDINLILATGGPGMVKAAYSSGKPAIGVGAGNTPVVIDETADIKRAVASILMSKTFDNGVICASEQSVIVVDSAYDAVRERFATHGGYMLKGKELHAVQGILLKNGSLNADIVGQPAPKIAEMAGITVPANTKVLIGEVTAVDESEPFAHEKLSPTLAMYRAKDFNDAVIKAEKLVAMGGIGHTSCLYTDQDNQPERVNHFGNMMKTARILINTPASQGGIGDLYNFKLAPSLTLGCGSWGGNSISENVGPKHLINKKTVAKRAENMLWHKLPKSIYFRRGSLPIALEEVASDGAKRAFIVTDRFLFNNGYVDQVTSVLKQHGLETEVFFEVEADPTLSIVRKGAEQMHSFKPDVIIALGGGSPMDAAKIMWVMYEHPTTHFEELALRFMDIRKRIYKFPKMGVKAKMVAITTTSGTGSEVTPFAVVTDDATGQKYPLADYALTPDMAIVDANLVMNMPKSLCAFGGLDAVTHSLEAYVSVLANEYSDGQALQALKLLKENLPDSYRDGAKNPVARERVHNAATIAGIAFANAFLGVCHSMAHKLGSEFHIPHGLANAMLISNVIRYNANDNPTKQTTFSQYDRPQARRRYAEIADHLRLTAPSDRTAQKIEKLLNWLEEIKTELGIPASIREAGVQEADFLAKVDKLSEDAFDDQCTGANPRYPLISELKQILLDTYYGRKFSEEVKTETVEPVAKAAKTGKKAAH

SEQ ID NO: 56 - protein, 878 aa, Salmonella typhimurium
MAVTNVAELNALVERVKKAQREYASFTQEQVDKIFRAAALAAADARIPLAKMAVAESGMGIVEDKVIKNHFASEYIYNAYKDEKTCGVLSEDDTFRTITIAEPIGIICGIVPTTNPTSTAIFKSLISLKTRNAIIFSPHPRAKEATNKAADIVSKALSLPARRKIRLARSINLPVELSNVDAPPGYIPDPATGSRHGYSCIQLGSTLSAIGQATLRLVLMKPLIDSNAACGLCLDAYNLCYRGNLCFCTSLFVVDSAYLHMRPDFRQHGGYMRRPELKAVQRYPEKWRSERAIVGQPAYKIAELAGFSVPETTKILIGEVTVVDESEPFAHEKLSPTLAMYRAKDFEEAVEKAEKLVAMGGIGHTSCLYTDQDNQPERVAYFGQMKNARILINTPASQGGIGDLYNFKLPPSLTLGCGSWGGNSISENVGPKHLIYKKTVAKRAENMWHKLPKSIYFRRGSLPIALDEVITDGHKRALIVTDRFCSTTVSDRSLCAERRGVETEVFFEVEAADPTLSVVRKGPELANSFKPDVIIAVGGVPRWTRGEIIGSCTNHPETRLIEERVRFMTSYRIYKFPKMVKAKCSPSTTTSGTGSKLHRLRLCPTTLLPQKYPLADYAVTPDMAIVDANLVMDMPNTLTRKGPLHRLTHAMERIVSVLASQSDGQALQALKEYFPASYHEGKNPVARERLHSAATIAPIAFANAFLGVCHWMAHKLPAQLHIPHGPFNARYRHSVRRAQSNPTKQVALSQYLYNFAAHRWPAERSIPRARTGIRPRKYKLVPVALCHVKGIKADLGIPKSIREAGVQEADFLAHVDKLSEDAFDDQCTGANPRYPLMSELKQILLDTYYGRDFTEGEVAAKKDVVAAPKAEKKAKKSA

SEQ ID NO: 57 - protein, 867 aa, Lactobacillus plantarum
MIKTEKNQTSKVTDEVDQLVQRSKKALAILKSYTQAQIDDLCEKVAVAALDNHMKLAKLAVEETGRGVVEDKAIKNIYASEYIWNSMRHDKTVGVIKEDDEEQLMEIAEPVGIVAGVTPVTNPTSTTVFKTLISLKGRNTIVFGFHPQAQKCSSAAADVMREAIKAAGGPADAVLYIEHPSIEATDALMHHTDVATILATGGPSMVTAAYSSGKPALGVGPGNGPTYVEKTADIKQAVNDIVLSKTFDNGMICASENSAIIDKEIYAEVKAEFIRLGCYYVKPKDVQALSDAVIDPNRHTVRGPVAGKTAYQIAQMAGLKDVAKDCRVLIAEINGVGIKYPLSGEKLSPVLTVYKADSHEAAFKRANELLHYGGLGHTAGIHTTDDALVKEFGLQMPACRILVNTPSSVGGLGNIYNNMAPSLTLGTGSYGGNSISHNVTDMDLINIKTVAKRRNNMQWVKMPPKVYFERNSVRYLEHMAGIKKVFLVCDPGMVEFGYADRVTAVLNKRTDPVDIDIFSEVEPNPSTDTVYKGVARMKAFKPDTIIALGGGSAMDAAKGMWLFYEHPEASFLGAKQKFLDIRKRTYKVPVSEKVTYIGIPTTSGTGSEVTPYAVITDSKTHVKYPITDYAMQPDIAIVDPQFVETVPKRTTAWTGLDVITHATEAYVSTMASDFTRGWSIQALQLAFKYLKASYDGDKMAREKMHNASTLAGMAFANAFLGINHSIAHKLGGEFNLPHGLAIAITYPQVVRYNAEIPTKLAMWPKYNHNTALADYANIARALGLPGKTDEELKESLVKAYIDLAHSMDVTLSLKANRVEKKHFDATVDELAELAYEDQCTTANPREPLISELKAIIEREWDGQGTEK

SEQ ID NO: 58 - protein, 903 aa, Lactococcus lactis
MATKKAAPAAKKVLSAEEKAAKFQEVVAYTDQLVKKAQAAVLKFEGYTQTQVDTIVAAMALAASKHSLELAHEAVNETGRGVVEDKDTKNHFASESVYNAIKNDKTVGVIAENKVAGSVEIASPLGVLAGIVPTTNPTSTAIFKSLLTAKTRNAIVFAFHPQAQKCSSHAAKIVYDAAIEAGAPEDFIQWIEVPSLDMTTALIQNRGIATILATGGPGMVNAALKSGNPSLGVGAGNGAVYVDATANIDRAVEDLLLSKRFDNGMICATENSAVIDASIYDEFVAKMPTQGAYMVPKKDYKAIESFVFVERAGEGFGVTGPVAGRSGQWIAEQAGVNVPKDKDVLLFELDKKNIGEALSSEKLSPLLSIYKSETREEGIEIVRSLLAYQGAGHNAAIQIGAMDDPFVKEYGIKVEASRILVNQPDSIGGVGDIYTDAMRPSLTLGTGSWGKNSLSHNLSTYDLLNVKTVAKRRNRPQWVRLPKEIYYEKNAISYLQELPHVHKAFIVADPGMVKFGFVDKVLEQLAIRPTQVETSIYGSVQPDPTLSEAIAIARQMNHFEPDTVICLGGGSALDAGKIGRLIYEYDARGEADLSDDASLKEIFQELAQKFVDIRKRIIKFYHPHKAQMVAIPTTSGTGSEVTPFAVITDDETHVKYPLADYQLTPQVAIVDPEFVMTVPKRTVSWSGIDAMSHALESYVSVMSSDYTKPISLQAIKLIFENLTESYHYDPAHPTKEGQKARENMHNAATLAGMAFANAFLGINHSLAHKIAGEFGLPHGLAIAIAMPHVIKFNAVTGNVKFTPYPRYETYRAQEDYAEISRFMGFAGKEDSDEKAVKALVAELKKLTDSIDINITLSGNGVDKAHLERELDKLADLVYDDQCTPANPRQPRIDEIKQLLLDQY